(19) United States
(12) Patent Application Publication
(10) Pub. No.: US 2018/0161003 A1
(43) Pub. Date: Jun. 14, 2018

(54) ULTRASOUND SIGNAL PROCESSING DEVICE, ULTRASOUND SIGNAL PROCESSING METHOD, AND ULTRASOUND DIAGNOSTIC DEVICE

(71) Applicant: Konica Minolta, Inc., Tokyo (JP)

(72) Inventor: Yasuhito Watanabe, Osaka-fu (JP)

(73) Assignee: Konica Minolta, Inc., Tokyo (JP)

(21) Appl. No.: 15/836,293

(22) Filed: Dec. 8, 2017

(30) Foreign Application Priority Data
Dec. 9, 2016 (JP) 2016-239550

(51) Int. Cl. A61B 8/00; A61B 8/08; G01S 15/89

(52) U.S. Cl. CPC A61B 8/44; A61B 8/4488; G01S 15/8961; G01S 15/8915; A61B 8/5207

(57) ABSTRACT

An ultrasound signal processing device includes ultrasound signal processing circuitry that operates as: a transmitter that causes an ultrasound probe to transmit ultrasound beams to an ultrasound main irradiation area defined by two straight lines each connecting a focal point and a different end of a transmission transducer element array; a receiver that generates receive signal sequences; a delay-and-sum calculator that sets first and second target areas in the ultrasound main irradiation area, and performs delay-and-summing of receive signal sequences based on ultrasound reflection from measurement points in the first and second target areas thereby to generate sub-frame acoustic line signals, the first target area is an entirety of an area located at or shallower than a focal depth, the second target area is part of an area located deeper than the focal depth; and a synthesizer that synthesizes sub-frame acoustic line signals to generate a frame acoustic line signal.

Japanese Patent Application No. 2016-239550 filed on Dec. 9, 2016, including description, claims, drawings, and abstract, is incorporated herein by reference in its entirety.

### TECHNICAL FIELD

The present invention is related to an ultrasound signal processing device, and an ultrasound diagnostic device equipped with the ultrasound signal processing device. In particular, the present invention relates to receive beam forming in an ultrasound signal processing device.

### BACKGROUND

Typically, an ultrasound diagnostic device transmits ultrasound towards the inside of a subject via an ultrasound probe (referred to in the following as a “probe”), and receives reflected ultrasound (an echo) via the probe. The reflected ultrasound is generated within the subject due to tissues in the subject having different acoustic impedances. Further, an ultrasound diagnostic device generates an ultrasound tomographic image based on electric signals acquired through the reception of the reflected ultrasound, and displays the ultrasound tomographic image on a monitor (referred to in the following as a “display unit”). An ultrasound tomographic image shows the structures of tissues inside the subject. Ultrasound diagnostic devices are widely used for the imaging diagnosis of subjects, for having low invasiveness and achieving real-time observation of tissues through tomographic images and the like.

A typical method applied in conventional ultrasound diagnostic devices for forming signals based on received reflected ultrasound (i.e., receive beam forming) is delay-and-sum beam forming. One example of delay-and-sum beam forming can be found disclosed in pages 42-45 of “Ultrasound Diagnostic Equipment,” written by Masayasu Itou and Tsuyoshi Mochizuki and published by Corona Publishing Co., Ltd (Aug. 26, 2002). According to this method, transmission beam forming (i.e., transmission of ultrasound by a plurality of transducer elements towards the inside of the subject) is typically performed such that a transmitted ultrasound beam converges (focuses) at a predetermined focal depth inside the subject. Further, according to this method, measurement points are always set along the central axis of the transmitted ultrasound beam. Due to this, one ultrasound transmission event generates only one or a few acoustic line signals along the central axis of the transmitted ultrasound beam, and thus, reflected ultrasound is not utilized in an efficient manner. In addition, with this method, an acoustic line signal acquired from a measurement point distant from the transmission focal point has low spatial resolution and low S/N ratio.

Meanwhile, a receive beam forming method is being proposed that utilizes a so-called synthetic aperture method to yield images with high spatial resolution and high quality not only from near the transmission focal point, but also, from areas other than near the transmission focal point. One example of receive beam forming utilizing the synthetic aperture method can be found disclosed in pages 395 through 405 of “Virtual Ultrasound Sources in High Resolution Ultrasound Imaging,” S. I. Nikolov and J. A. Jensen, in Proc, SPIE—Progress in Biomedical Optics and Imaging, Vol. 3, 2002. According to this method, delaying is performed taking into consideration both a propagation path of ultrasound and the time amount required for reflected ultrasound to arrive at a transducer element by travelling along the propagation path. Thus, the method achieves receive beam forming making use of not only reflected ultrasound from an area of an ultrasound main irradiation area near the transmission focal point, but also, reflected ultrasound from areas of the ultrasound main irradiation area other than the area near the transmission focal point. Due to this, the method enables generating, from one ultrasound transmission event, acoustic line signals covering the entire ultrasound main irradiation area, including areas far from the transmission focal point. Note that in the present disclosure, an ultrasound main irradiation area is an area such that at every point in the ultrasound main irradiation area, ultrasound transmitted from transducer elements composing a transmission transducer element array is in-phase. In addition, the synthetic aperture method enables setting a virtual transmission focal point with respect to each measurement point based on multiple receive signals acquired for the same measurement point through multiple transmission events. Thus, the synthetic aperture method enables acquiring an ultrasound image with higher spatial resolution and higher S/N ratio than the receive beam forming method disclosed in “Ultrasound Diagnostic Equipment.”

In the synthetic aperture method, for efficient use of ultrasound and high resolution, a large size area for which acoustic line signals for a single transmission event are generated (referred to in the following as a “target area”) is used, with the entire ultrasound main irradiation area used as the target area. However, an increase in target area size brings about a proportional increase in the number of measurement points in the target area and an increase in computation amount for delay-and-summing taking into consideration transmission and reception delays. Due to this, an increase in target area size necessitates hardware with high computation capability to achieve high-speed delay-and-sum computation, and thus ultrasound diagnostic device cost is increased. Meanwhile, when simply reducing target area size, improvement of spatial resolution and S/N ratio becomes insufficient.

### SUMMARY

One or more embodiments of the present invention provide an ultrasound signal processing device that enables reducing delay-and-sum computation amount in a synthetic aperture method utilizing converging-type transmission beam forming while suppressing decrease in spatial resolution and S/N ratio, and an ultrasound diagnostic device including the ultrasound signal processing device.

To achieve the abovementioned aim, an ultrasound signal processing device reflecting one aspect according to one or more embodiments of the present invention are ultrasound signal processing devices that performs multiple transmission events of transmitting converging ultrasound beams to a subject by using an ultrasound probe having multiple transducer elements, that performs, for each of the transmission events, reception of ultrasound reflection from the subject and generation of a sub-frame acoustic line signal based on the ultrasound reflection, and that synthesizes sub-frame acoustic line signals for the respective transmission events to generate a frame acoustic line signal, the ultrasound signal processing device comprising ultrasound signal processing circuitry configured to operate as: a transmitter that, for each of the transmission events, while shifting a transmission transducer element array of the ultrasound probe in a transducer element array direction in which the transducer elements are arrayed, causes the ultrasound probe to transmit ultrasound beams to an ultrasound main irradiation area by using the transmission transducer element array, the ultrasound beams converging at a focal point defined by a position of the transmission transducer element array, the ultrasound main irradiation area being defined as an area positioned between two straight lines each connecting the focal point and a different end of the transmission transducer element array; a receiver that, for each of the transmission events, generates sequences of receive signals for receive transducer elements of the ultrasound probe based on ultrasound reflection that the ultrasound probe receives from the subject; a delay-and-sum calculator that, for each of the transmission events, sets a first target area and a second target area included in the ultrasound main irradiation area, and performs delay-and-summing of sequences of receive signals based on ultrasound reflection from measurement points located in the first target area and the second target area thereby to generate a sub-frame acoustic line signal for each of the measurement points, the first target area being entirety of an area located at or shallower than a focal depth where the focal point is located, the second target area being part of an area located deeper than the focal depth; and a synthesizer that synthesizes sub-frame acoustic line signals for the transmission events to generate a frame acoustic line signal.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

In the drawings:

FIG. 1 is a functional block diagram illustrating the structure of an ultrasound diagnostic device 100 pertaining to one or more embodiments;

FIG. 2 is a schematic illustrating a propagation path of ultrasound beam transmitted from a transmission beam former 103 pertaining to one or more embodiments;

FIG. 3 is a functional block diagram illustrating the structure of a receive beam former 104 pertaining to one or more embodiments;

FIG. 4 is a functional block diagram illustrating the structure of a delay-and-sum calculator 1041 pertaining to one or more embodiments;

FIG. 5 is a schematic illustrating a target area Bx pertaining to one or more embodiments;

FIG. 6 is a schematic illustrating the relationship between a transmission aperture Tx and a receive aperture Rx set by a receive aperture setter 1043 pertaining to one or more embodiments;

FIG. 7A is a schematic pertaining to one or more embodiments, illustrating one propagation path of ultrasound that is transmitted from the transmission aperture Tx and arrives at a receive transducer element Rk via a measurement point Pij; FIG. 7B is a schematic pertaining to one or more embodiments, illustrating another propagation path of ultrasound that is transmitted from the transmission aperture Tx and arrives at a receive transducer element Rk via a measurement point Pij;

FIG. 8 is a functional block diagram illustrating the structure of a synthesizer 1140 pertaining to one or more embodiments;

FIG. 9 is a schematic illustrating processing by an adder 11401 pertaining to one or more embodiments for generating a synthesized acoustic line signal;

FIGS. 10A and 10B are schematics pertaining to one or more embodiments, providing an overview of maximum overlap counts of synthesized acoustic line signals and amplification by an amplifier 11402;

FIG. 11 is a flowchart illustrating beam forming by the receive beam former 104 pertaining to one or more embodiments;

FIG. 12 is a flowchart illustrating operations of the receive beam former 104 pertaining to one or more embodiments for generating an acoustic line signal for a measurement point Pij;

FIG. 13 is a schematic for explaining the operations of the receive beam former 104 pertaining to one or more embodiments for generating an acoustic line signal for a measurement point Pij;

FIG. 14 is a schematic illustrating the relationship between a transmission aperture Tx and a receive aperture Rx set by a Tx receive aperture setter pertaining to modification 1 of one or more embodiments;

FIG. 15 is a flowchart illustrating beam forming by a receive beam former of an ultrasound diagnostic device pertaining to modification 1 of one or more embodiments;

FIG. 16 is a schematic for explaining the operations of the receive beam former pertaining to modification 1 for generating an acoustic line signal for a measurement point Pij;

FIG. 17 illustrates a first setting example of a target area Bx pertaining to modification 2 of one or more embodiments;

FIG. 18 illustrates a second setting example of the target area Bx pertaining to modification 2 of one or more embodiments; and

FIGS. 19A and 19B illustrate an evaluation image and a target area Bx pertaining to one or more embodiments.

### DETAILED DESCRIPTION

Hereinafter, one or more embodiments of the present invention will be described with reference to the drawings. However, the scope of the invention is not limited to the disclosed embodiments.

The inventor conducted various considerations for reducing computation amount while suppressing a decrease in spatial resolution and S/N ratio of acoustic line signals (referred to in the following as “acoustic line signal quality”) in an ultrasound diagnostic device deploying a synthetic aperture method.

Typically, converging-type transmission beam forming is performed by causing a wavefront to converge so that an ultrasound beam focuses at a certain depth of a subject (referred to in the following as a “focal depth”). In each transmission of ultrasound (transmission event), transducer elements that are used for ultrasound transmission (referred to in the following as a “transmission transducer element array”) mainly transmit ultrasound to the ultrasound main irradiation area. For example, when ultrasound transmission is performed with one measurement point set as the transmission focal point, the ultrasound main irradiation area has an hourglass shape, the bottom edge (i.e., base) of the ultrasound main irradiation area corresponds to the transmission transducer element array, and two straight lines each extending from a different end of the base towards the transmission focal point partition the ultrasound main irradiation area from the outside thereof. Further, the wavefront of ultrasound transmitted from the transmission transducer element array forms an arc, being a segment of a circle whose center corresponds to the transmission focal point. Here, it should be noted that ultrasound beams do not always converge (i.e., focus) to a single point as described above. For example, ultrasound beams may converge to an area having a width corresponding to 1.5 times the width of a single transducer element to several times the width of a single transducer element. When ultrasound beams converge at such an area, the width of the ultrasound main irradiation area in a direction in which transducer elements are arrayed (referred to in the following as a transducer element array direction) decreases as approaching the transmission focal depth, equals the width of the transmission focal area in the transducer element array direction at the transmission focal depth, and increases in the transducer element array direction once again as departing the transmission focal depth towards deeper areas. For convenience of description, a center point of the focal area at the focal depth in such a case is referred to as a “focal point.” That is, regardless of whether or not ultrasound beams focus at a single point, the ultrasound main irradiation area converges, at the focal depth, at the focal point or at the focal area, which is an area including the focal point and the vicinity of the focal point. Meanwhile, at depths other than the focal depth, the greater the distance from the focal depth, the greater the width of the ultrasound main irradiation area in the transducer element array direction.

Further, with the synthetic aperture method, for each transmission event, measurement points can be set to cover the entire ultrasound main irradiation area of the transmission event. As such the entirety of the ultrasound main irradiation area is set as a target area. Meanwhile, a target area for one transmission event cannot cover the entirety of an area corresponding to one frame image (referred to in the following as a “region of interest (ROI)”). As such, a plurality of transmission events, for each of which a different target area is set, need to be conducted to generate one frame ultrasound image. Taking this into consideration, for efficient use of ultrasound, a target area for a single transmission event covers as great an area of an ultrasound main irradiation area for the transmission event. Further, in general, to improve spatial resolution and signal S/N ratio, the target areas for two consecutive transmission events should overlap one another as much as possible.

However, the number of measurement points included in a target area is proportional to target area size. Consequently, computation amount for delay-and-summing and the memory amount necessary to store acoustic line signals produced through the delay-and-summing are proportional to target area size. Due to this, an increase in target area size directly results in an increase in ultrasound diagnostic device memory amount required. Further, when ultrasound diagnostic device computation capability is insufficient with respect to delay-and-summing computation amount, a decrease in temporal resolution and usability may occur. This is because ultrasound diagnostic devices are not capable of achieving frame rate higher than that corresponding to their computation capability, and thus a decrease in ultrasound image frame rate may occur. Accordingly, in order to suppress such decrease in temporal resolution and usability, a processor with computation capability high enough to perform delay-and-summing computation at high speed, such as a high performance GPU, becomes necessary, which leads to an increase in ultrasound diagnostic device cost.

One measure that can be considered for reducing computation amount is reducing the number of measurement points included in the target area. Possible measures for reducing the number of measurement points include reducing target area size and reducing measurement point density in the target area. However, when reducing target area size in the depth direction, the area for which an ultrasound image can be generated decreases in proportion with target area size. Further, when reducing measurement point density in the depth direction, distance resolution, which is spatial resolution in the depth direction, decreases proportionally. Hence, the inventor sought for a method of reducing the number of measurement points in the transducer element array direction while suppressing a decrease in acoustic line signal quality, and arrived at the idea of partitioning a target area into a first target area located at or shallower than the focal depth and a second target area located deeper than the focal depth, and reducing width or density of measurement points in the transducer element array direction only with respect to the second target area. By making such a configuration, the number of measurement points can be reduced without reducing the number of measurement points or the density of measurement points in the depth direction. Due to this, neither distance resolution nor the area for which an ultrasound image is generated decreases. Further, by reducing the number of measurement points in an area including many measurement points for which a high S/N ratio is not achieved, it is possible to reduce computation amount while suppressing decrease in S/N ratio of the entire acoustic line signals. In areas deeper than the focal depth, since ultrasound attenuation increases as departing the focal point, S/N ratio is lower than in areas shallower than the focal depth. Thus, a small influence is exerted on the deeper areas in case of decrease in S/N ratio and spatial resolution due to decrease in the number of synthesizing. Meanwhile, in the ultrasound main irradiation area, since the width in the transducer element array direction increases as departing the focal depth, the number of measurement points increases as departing the focal point. Thus, by reducing the number of measurement points in the second target area, it is possible to reduce computation amount in accordance with a reduction amount of the number of measurement points.

The following one or more embodiments describe an ultrasound signal processing method and an ultrasound diagnostic device including the ultrasound signal processing method in detail, with reference to the accompanying drawings.

### Overall Structure

The following describes an ultrasound diagnostic device 100 pertaining to one or more embodiments, with reference to the accompanying drawings.

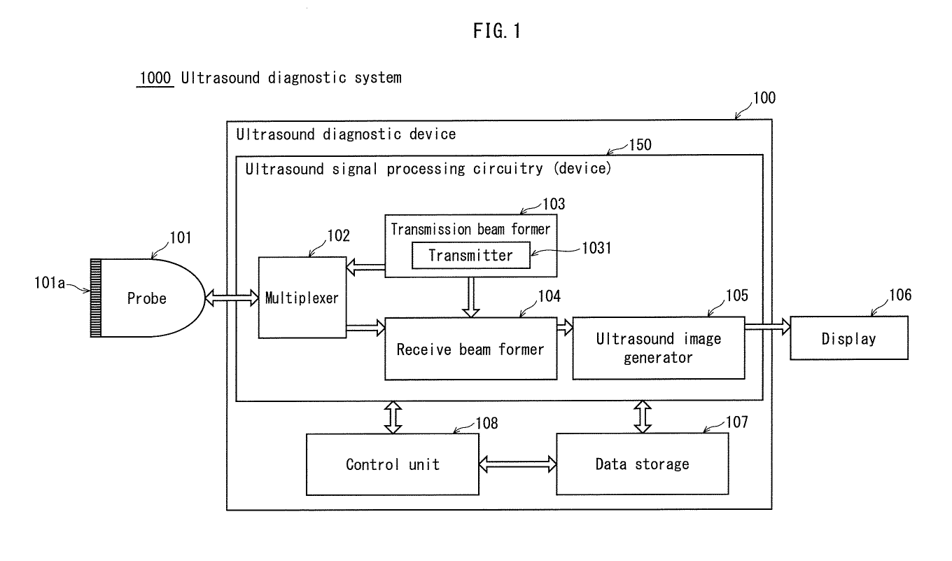

FIG. 1 illustrates functional blocks of an ultrasound diagnostic system 1000 pertaining to one or more embodiments. As illustrated in Fig. I, the ultrasound diagnostic system 1000 includes: a probe 101; the ultrasound diagnostic device 100; and a display unit 106. The probe 101 includes a plurality of transducer elements 101a. Each of the transducer elements 101a is capable of transmitting ultrasound towards the subject and receiving reflected ultrasound (echo signals). The ultrasound diagnostic device 100 causes the probe 101 to perform transmission/reception of ultrasound, and generates an ultrasound image based on signals output from the probe 101. The display unit 106 displays the ultrasound image on any display device provided thereto. The probe 101 and the display unit 106 are separately connectable to the ultrasound diagnostic device 100. FIG. 1 illustrates the ultrasound diagnostic device 100 with the probe 101 and the display unit 106 connected thereto. Alternatively, the ultrasound diagnostic device 100 may include therein the probe 101 and the display unit 106.

### Structure of Ultrasound Diagnostic Device 100

The ultrasound diagnostic device 100 includes a multiplexer 102; a transmission beam former 103; and a receive beam former 104. The multiplexer 102 selects one or more of the transducer elements 101a for ultrasound transmission and one or more of the transducer elements 101a for ultrasound reception. The multiplexer 102 provides each of the transducer elements 101a for ultrasound transmission with input, and receives output from the transducer elements 101a for ultrasound reception. The transmission beam former 103 controls timings of application of a high voltage for ultrasound transmission to each of the transducer elements 101a for ultrasound transmission. The receive beam former 104 performs some amplification and A/D conversion on electric signals yielded by the transducer elements 101a for ultrasound reception, based on reflected ultrasound received by the probe 101, and performs receive beam forming to generate acoustic line signals. In addition, the ultrasound diagnostic device 100 includes an ultrasound image generator 105; a data storage 107; and a control unit 108. The ultrasound image generator 105 generates an ultrasound image (a B-mode image) based on signals output from the receive beam former 104. The data storage 107 stores the acoustic line signal output from the receive beam former 104 and the ultrasound image output from the ultrasound image generator 105. The control unit 108 controls each of the other components of the ultrasound diagnostic device 100.

Among the components of the ultrasound diagnostic device 100, the multiplexer 102, the transmission beam former 103, the receive beam former 104, and the ultrasound image generator 105 constitute ultrasound signal processing circuitry 150, and the ultrasound signal processing circuitry 150 constitutes an ultrasound signal processing device.

Each component of the ultrasound diagnostic device 100, for example, each of the multiplexer 102, the transmission beam former 103, the receive beam former 104, the ultrasound image generator 105, and the control unit 108 may be implemented by using a hardware circuit such as a field-programmable gate array (FPGA), an application-specific integrated circuit (ASIC), or the like. Alternatively, each of the components may be implemented by using a combination of software and a programmable device such as a processor. As a processor, a central processing unit (CPU) or a graphics processing unit (GPU) may be used for example, and a construction using a GPU is referred to as a General-purpose computing on graphics processing unit (GPGPU). Each of such components may be implemented as one circuit component, or as an aggregate of a plurality of circuit components. Further, a plurality of such components may be implemented by using one circuit component, or as an aggregate of a plurality of circuit components.

The data storage 107 is a computer-readable recording medium. For example, the data storage 107 may be implemented by using a flexible disk, a hard disk, an MO, a DVD, a DVD-RAM, a BD, or a semiconductor memory. Alternatively, the data storage 107 may be an external storage device connected to the ultrasound diagnostic device 100.

Note that the ultrasound diagnostic device 100 pertaining to one or more embodiments need not have the structure illustrated in FIG. 1. For example, the ultrasound diagnostic device 100 may not include the multiplexer 102, and the transmission beam former 103 and the receive beam former 104 may be directly connected with each transducer element 101 a of the probe 101. Further, the probe 101 may have built-in therein a part or the entirety of each of the transmission beam former 103, the receive beam former 104, and the like. Such modifications apply not only to the ultrasound diagnostic device 100, but also similarly apply to the ultrasound diagnostic devices of one or more embodiments described later in the present disclosure.

### Structure of Main Part of Ultrasound Diagnostic Device 100

The ultrasound diagnostic device 100 pertaining to one or more embodiments is characterized for including the transmission beam former 103 and the receive beam former 104. The transmission beam former 103 causes the transducer elements 101a of the probe 101 to transmit ultrasound beam. The receive beam former 104 performs computation with respect to electric signals acquired through the reception of reflected ultrasound in the probe 101, and generates acoustic line signals used in forming an ultrasound image. Accordingly, the present disclosure focuses on the structure and the functions of each of the transmission beam former 103 and the receive beam former 104. Note that components other than the transmission beam former 103 and the receive beam former 104 may have structures and functions similar to those in conventional ultrasound diagnostic devices. In other words, the ultrasound diagnostic device 100 may be implemented by replacing beam formers in a conventional ultrasound diagnostic device with the beam formers pertaining to one or more embodiments.

The following describes the structure of each of the transmission beam former 103 and the receive beam former 104.

### 1. Transmission Beam Former 103

The transmission beam former 103 is connected to the probe 101, via the multiplexer 102. However, note that the multiplexer 102 is not a mandatory element in one or more embodiments of the present invention. The transmission beam former 103 controls timings of application of high voltage with respect to each of a plurality of transducer elements 101a composing a transmission aperture Tx. The transmission aperture Tx is an array of transducer elements composed of all or some of the transducer elements 101a of the probe 101. Note that in the following, the term “transmission transducer element” is used to refer to transducer elements composing the transmission aperture Tx. The transmission beam former 103 includes a transmitter 1031.

The transmitter 1031 performs transmission processing. The transmission processing involves supplying a transmission signal having a pulsar waveform to each of the transmission transducer elements. A transmission transducer element receiving a transmission signal transmits an ultrasound beam. The transmitter 1031 supplies transmission signals to the transmission transducer elements based on transmission control signals output from the control unit 108. In specific, the transmitter 1031 includes, for example, a clock generation circuit, a pulse generation circuit, and a delay circuit. The clock generation circuit generates a clock signal specifying the transmission timing of ultrasound beams. The pulse generation circuit generates pulse signals for driving the transmission transducer elements. The delay circuit performs focus processing so that ultrasound beams are appropriately focused. In specific, the delay circuit sets a delay time for each transmission transducer element, and delays the transmission of the ultrasound beam from the transmission transducer element by the corresponding delay time.

The transmitter 1031 repetitively performs ultrasound transmission while shifting the transmission aperture Tx by a shift pitch Mp in the transducer element array direction each time, so that all of the transducer elements 101a of the probe 101 transmit ultrasound. In one or more embodiments, the shift pitch Mp corresponds to width of a single transducer element, and transmission apertures Tx corresponding to two consecutive transmission events differ in position in the transducer element array direction by an amount corresponding to the width of a single transducer element. Note that the shift pitch Mp is not limited to the width of a single transducer element, and alternatively may correspond to width of one-half of a single transducer element. Further, the transmitter 1031 outputs information indicating the positions of transmission transducer elements composing the transmission aperture Tx to the data storage 107, via the control unit 108. For example, supposing that the probe 101 has one hundred and ninety-two (192) transducer elements 101a in total, the number of transmission transducer elements composing the transmission aperture Tx may be twenty (20) to one hundred (100). Further, in the present disclosure, the term transmission event is used to refer to ultrasound transmission by the transmitter 1031, performed by using one transmission aperture (i.e., one set of transmission transducer elements of the predetermined number).

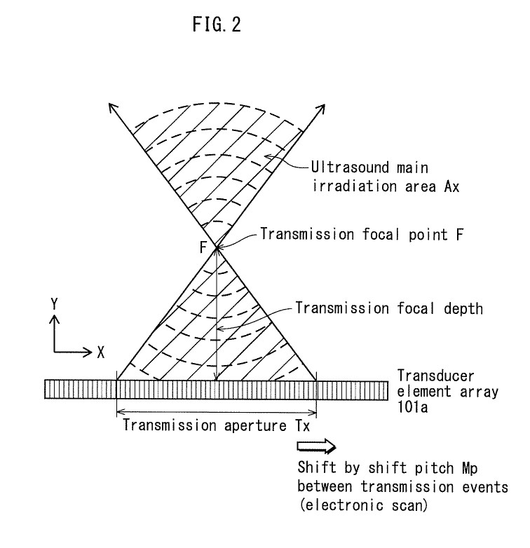

FIG. 2 is a schematic illustrating a propagation path of ultrasound transmitted by the transmission beam former 103. FIG. 2 illustrates a transmission aperture Tx for one transmission event (i.e., a transmission transducer element array composed of transmission transducer elements 101a that contribute to ultrasound transmission in the transmission event). Further, the transmission-array direction length of the transmission aperture Tx is considered the length of the transmission aperture Tx.

The transmission beam former 103 controls ultrasound transmission by the transmission transducer elements such that a transmission transducer element closer to the center position of the transmission aperture Tx transmits ultrasound later in the transmission event. Due to this, the wavefront of ultrasound transmitted from the transmission transducer elements composing the transmission aperture Tx converges at one point at a certain focal depth in the subject (i.e., the transmission focal point F). Note that the depth of the transmission focal point F (i.e., transmission focal depth) can be set. Here, the focal depth indicates a depth at which the largest amount of transmitted ultrasound converges in the transducer element array direction (X direction in FIG. 2), in other words, a depth at which the width of ultrasound beams in the X direction is the narrowest. The focal point F is a center position of ultrasound beams in the X direction at the focal depth. Note that the focal depth is constant for transmission events of a single frame. In other words, no change occurs in a relative relationship between the transmission aperture Tx and the focal point F for transmission events of a single frame. After converging at the transmission focal point F, the wavefront of the transmitted ultrasound spreads out as before converging at the transmission focal point F. Thus, the transmitted ultrasound propagates through an hourglass-shaped area whose base is defined by the transmission aperture Tx and which is partitioned from other areas inside the subject by two straight lines intersecting at the transmission focal point F. More specifically, ultrasound transmitted from the transmission aperture Tx propagates in the following manner. As the transmitted ultrasound advances in a depth direction of the subject from the transmission aperture Tx, the width thereof (length along horizontal axis (X axis) in FIG. 2) gradually decreases until reaching the minimum width at the transmission focal point F. Then, as the transmitted ultrasound advances further in the depth direction from the transmission focal point F (i.e., as the ultrasound advances in the upward direction in FIG. 2), the width thereof increases (i.e., the ultrasound spreads out). In the following, the hourglass-shaped area described above is referred to as an ultrasound main irradiation area Ax. Note that as already described above, the transmission of ultrasound may be performed so that the ultrasound main irradiation area Ax converges at the focal area.

### 2. Receive Beam Former 104

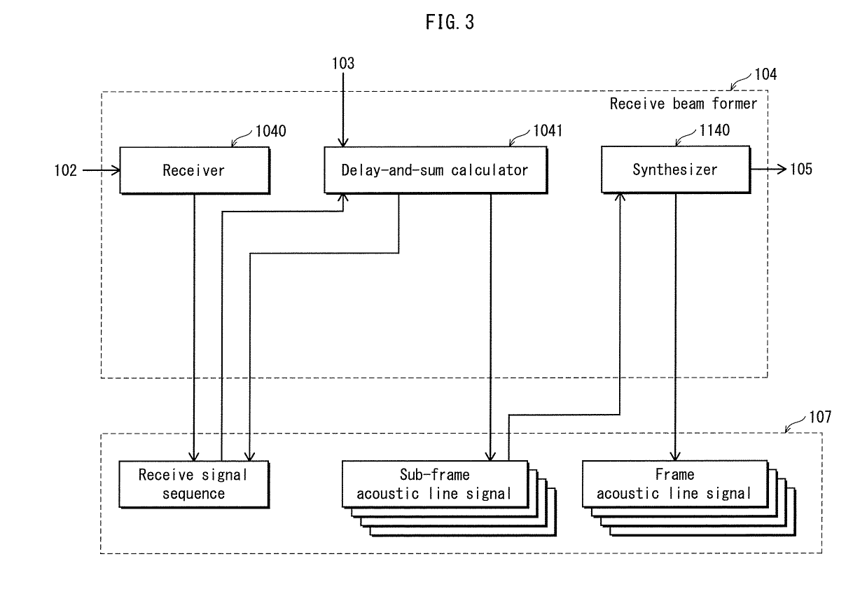

The receive beam former 104 generates acoustic line signals from electric signals acquired by a plurality of transducer elements 101a. The transducer elements 101a acquire the electric signals based on reflected ultrasound received by the probe 101. Here, an acoustic line signal for one measurement point is generated by performing delay-and-sum processing with respect to receive signals from the measurement point. Description of the delay-and-sum processing is provided later in the present disclosure. FIG. 3 is a functional block diagram illustrating the structure of the receive beam former 104. As illustrated in FIG. 3, the receive beam former 104 includes: a receiver 1040; a delay-and-sum calculator 1041; and a synthesizer 1140.

The following describes the structure of each functional block of the receive beam former 104.

### (1) Receiver 1040

The receiver 1040 is connected to the probe 101, via the multiplexer 102. However, note that the multiplexer 102 is not a mandatory element in one or more embodiments of the present invention. For each transmission event, the receiver 1040 generates receive signals (RF signals). The receiver 1040 generates the receive signals by first amplifying electric signals acquired through the probe 101 receiving reflected ultrasound, and then performing A/D conversion on the amplified signals. The receiver 1040 performs the generation of receive signals for each transmission event, and outputs the receive signals to be stored in the data storage 107.

Here, the receiver 1040 generates one receive signal sequence (RF signal) for each of some or all of the transducer elements 101a of the probe 101. In specific, a receive signal sequence for a given transducer element is a digital signal yielded by performing A/D conversion on an electrical signal yielded through conversion of reflected ultrasound received by the transducer element, and is a sequence of signals along the ultrasound transmission direction (corresponding to the depth direction) that are received by the transducer element.

As discussed above, in each transmission event, the transmitter 1031 causes the plurality of transmission transducer elements composing the transmission aperture Tx, among the transducer elements 101a of the probe 101, each to transmit an ultrasound beam. Meanwhile, for each ultrasound transmission event, the receiver 1040, based on ultrasound reflection that each of some or all of the plurality of transducer elements 101a of the probe 101 acquires from the transmission event, generates a receive signal sequence for each of the transducer elements 101a having acquired the ultrasound reflection. In the present disclosure, the transducer elements 101a acquiring ultrasound reflection are referred to as “reception transducer elements.” Here, the number of reception transducer elements may be greater than the number of transmission transducer elements composing the transmission aperture Tx. Further, the number of reception transducer elements may be equal to the total number of transducer elements 101a of the probe 101.

Further, as already discussed above, the transmitter 1031 repetitively performs transmission events while shifting the transmission aperture Tx by the shift pitch Mp in the transducer element array direction each time, so that all of the transducer elements 101a of the probe 101 transmit ultrasound. Meanwhile, for each ultrasound transmission event, the receiver 1040 generates receive signal sequences for reception transducer elements 101a, and stores the receive signal sequences to the data storage 107.

### (2) Delay-and-Sum Calculator 1041

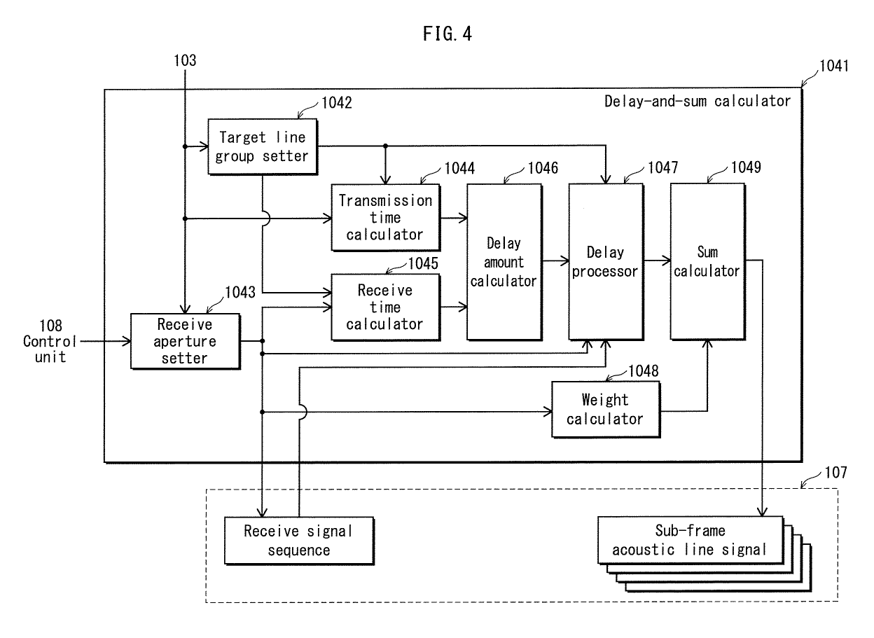

The delay-and-sum calculator 1041 is a circuit that sets a target area Bx for each transmission event. A target area Bx is an area in the subject from which one sub-frame acoustic line signal is to be generated, and is composed of target lines on which measurement points Pij are located. Further, the delay-and-sum calculator 1041 performs, for each measurement point Pij of the target area Bx, delay-and-sum processing with respect to receive signal sequences corresponding to the measurement point Pij, each of which is received by one receive transducer element Rk. The delay-and-sum calculator 1041 performs this processing for each transmission event having been performed. The delay-and-sum calculator 1041, for each transmission event, generates a sub-frame acoustic line signal for the transmission event by calculating an acoustic line signal for each measurement point of the target area Bx for the transmission event. FIG. 4 is a functional block diagram illustrating the structure of the delay-and-sum calculator 1041. As illustrated in FIG. 4, the delay-and-sum calculator 1041 includes: a target area setter 1042; a receive aperture setter 1043; a transmission time calculator 1044; a receive time calculator 1045; a delay amount calculator 1046; a delay processor 1047; a weight calculator 1048; and a sum calculator 1049.

The following describes the structure of each functional block of the delay-and-sum calculator 1041.

### i) Target Area Setter 1042

The target area setter 1042 sets the target area Bx, which is an area in the subject from which one sub-frame acoustic line signal is to be generated. More specifically, in the present disclosure, the term “target area” is used to indicate a signal area for generating a sub-frame acoustic line signal for one transmission event. Further, one acoustic line signal is generated for each measurement point Pij of the target area Bx. In other words, the target area Bx is set for each transmission event in order to specify ones of the measurement points for which acoustic line signals are to be generated for the transmission event.

Further, in the present disclosure, a sub-frame acoustic line signal is a group of acoustic lines signals that are generated from one transmission event. As already described above, from one transmission event, a plurality of acoustic line signals are generated, each for a different one of the measurement points Pij of the target area Bx. Further, a sub-frame is a unit corresponding to a group of signals which are acquired from one transmission event and each of which corresponds to a different one of the measurement points Pij of the target area Bx for the transmission event. Thus, a synthesizing result of multiple sub-frames acquired at different time points equals one frame.

For each transmission event, the target area setter 1042 sets the target area Bx based on the information indicating the position of the transmission aperture Tx for the transmission event, which is acquired from the transmission beam former 103.

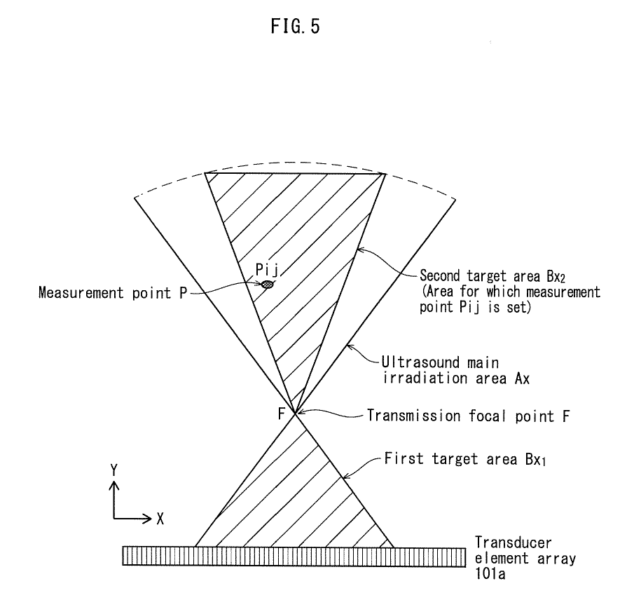

FIG. 5 is a schematic illustrating one example of the target area Bx. As illustrated in FIG. 5, the target area Bx is located in the ultrasound main irradiation area Ax, and includes a first target area Bx₁ located at or shallower than the focal depth and a second target area Bx₂ located deeper than the focal depth. The first target area Bx₁ is set so as to be the entirety of an area located at or shallower than the focal depth in the ultrasound main irradiation area Ax. Meanwhile, the second target area Bx₂ is set so as to be part of an area located deeper than the focal depth in the ultrasound main irradiation area Ax and to have a small width in the transducer element array direction. More specifically, the first target area Bx₁ is for example an isosceles triangle having the transmission aperture Tx as a bottom and the focal point F as a vertex. Meanwhile, the second target area Bx₂ is for example an isosceles triangle having a direct line parallel to the transducer element array direction at a certain depth as a bottom and the focal point F as a vertex. Here, when an inner angle at the focal point F in the first target area Bx₁ is represented by θ₁ and an inner angle at the focal point F in the second target area Bx₂ is represented by θ₂, the following relation is satisfied.

tan(θ₁/2)=n·tan(θ₂/2) (θ₁>θ₂, 1>n>0)

Here, when the focal depth is represented by Df, a width of the second target area Bx₂ in the transducer element array direction at a depth Df+d is narrower than a width of the first target area Bx₁ in the transducer element array direction at a depth Df−d, and specifically is n times the width of the first target area Bx₁ in the transducer element array direction at the depth Df−d. Moreover, the first target area Bx₁ and the second target area Bx₂ each have the central axis that coincides the central axis of the ultrasound main irradiation area Ax. Note that the shape of the second target area Bx₂ is not limited to the above example. The second target area Bx₂ may have any arbitrary shape as long as the following relationship is satisfied that the width in the transducer element array direction of the second target area Bx₂ at the depth Df+d is smaller than the width in the transducer element array direction of the first target area Bx₁ at the depth Df−d. Note that the first target area Bx₁ may be not the entirety but part of the area located at or shallower than the focal depth in the ultrasound main irradiation area Ax. Also, the focal point F may be included not in the first target area Bx₁ but in the second target area Bx₂. With this configuration, with respect to areas located at or shallower than the focal depth, it is possible to improve the use efficiency of ultrasound irradiation on measurement points set over substantially the entirety of the ultrasound main irradiation area Ax. Further, with respect to areas deeper than the focal depth, it is possible to reduce computation amount while reducing the number of measurement points in the transducer element array direction to suppress an influence of decrease in acoustic line signal quality.

The target area setter 1042 outputs the target area Bx to the receive aperture setter 1043, the transmission time calculator 1044, the receive time calculator 1045, and the delay processor 1047.

### ii) Receive Aperture Setter 1043

The receive aperture setter 1043 is a circuit that sets, for each transmission event, receive apertures Rx based on a control signal from the control unit 108 and information from the target area setter 1042 indicating the target area Bx for the transmission event. In specific, the receive aperture setter 1043 selects, for each measurement point Pij of the target area Bx, some of the transducer elements 101a of the probe 101 as receive transducer elements forming a transducer element array (referred to in the following as a receive transducer element array) whose center position corresponds to a transducer element Xk spatially closest to the measurement point Pij.

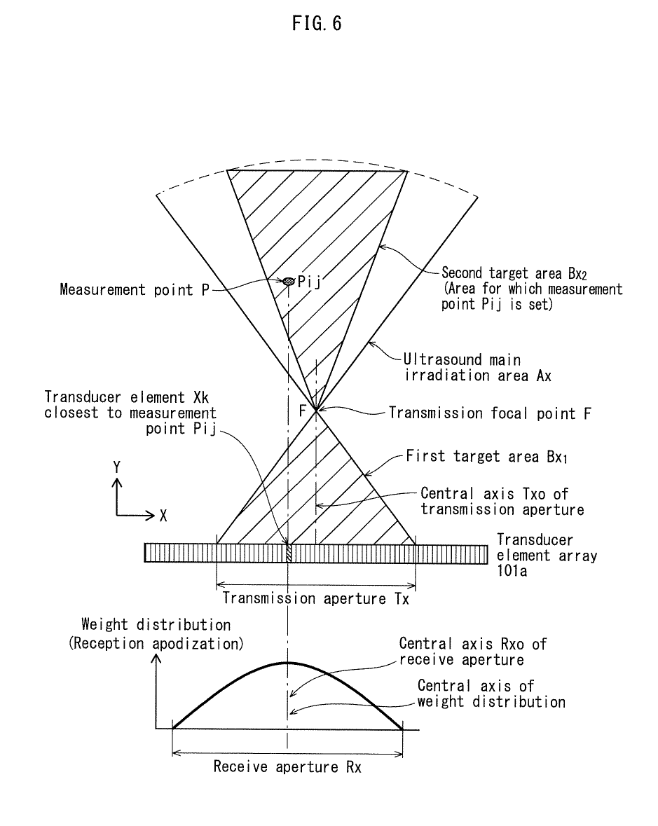

The receive aperture setter 1043 sets, for each measurement point Pij of the target area Bx for a transmission event, a receive aperture Rx (i.e., the receive transducer element array) so that the center position of the receive aperture Rx in the transducer element array direction corresponds to a transducer element Xk that is spatially closest to the measurement point Pij. FIG. 6 is a schematic illustrating the relationship between a transmission aperture Tx and a receive aperture Rx that the receive aperture setter 1043 sets. As illustrated in FIG. 6, for a given measurement point Pij, the receive aperture Rx is set so that the center position of the receive aperture Rx in the transducer element array direction corresponds to a transducer element Xk that is spatially closest to the measurement point Pij. Due to this, the position of the receive aperture Rx depends upon the position of the measurement point Pij, and does not change depending upon the position of the transmission aperture Tx, which shifts each time a transmission event is performed. That is, delay-and-sum processing for generating an acoustic line signal for a given measurement point Pij is always performed based on receive signal sequences acquired by receive transducer elements Rk composing the same receive aperture Rx. This means that with respect to the measurement point Pij, the same receive aperture Rx is used in delay-and-sum processing irrespective of transmission events.

In order to utilize reflected ultrasound from the entirety of the ultrasound main irradiation area, the number of the receive transducer elements composing each receive aperture Rx is, beneficially, greater than or equal to the number of transmission transducer elements composing each transmission aperture Tx. For example, the number of receive transducer elements may be 32, 64, 96, 128, 192, and so on.

The setting of the receive apertures Rx is performed at least by the number that is equal to the maximum number of measurement points Pij in the transducer element array direction. Further, the setting of receive apertures Rx may be performed each time a transmission event is performed as described above, or alternatively, receive apertures Rx for multiple transmission events having been performed may be set at once after the completion of the transmission events.

Further, the receive aperture setter 1043 outputs information indicating the positions of the receive transducer elements composing the receive aperture Rx to the data storage 107, via the control unit 108.

The data storage 107 outputs the information indicating the positions of the receive transducer elements composing the receive aperture Rx along with receive signal sequences for the receive transducer elements to each of the transmission time calculator 1044, the receive time calculator 1045, the delay processor 1047, and the weight calculator 1048.

### iii) Transmission Time Calculator 1044

The transmission time calculator 1044 is a circuit that, for each transmission event, calculates a transmission time for each measurement point P of the target area Bx for the transmission event. The transmission time for a given measurement point P is the time amount required for transmitted ultrasound to arrive at the measurement point P. The transmission time calculator 1044 acquires information indicating the positions of the transmission transducer elements for a given transmission event from the data storage 107, and information indicating the position of the target area Bx for the transmission event from the target area setter 1042. Based on such information, the transmission time calculator 1044, for each measurement point Pij located in the target area Bx, calculates the transmission time required for transmitted ultrasound to arrive at the measurement point Pij.

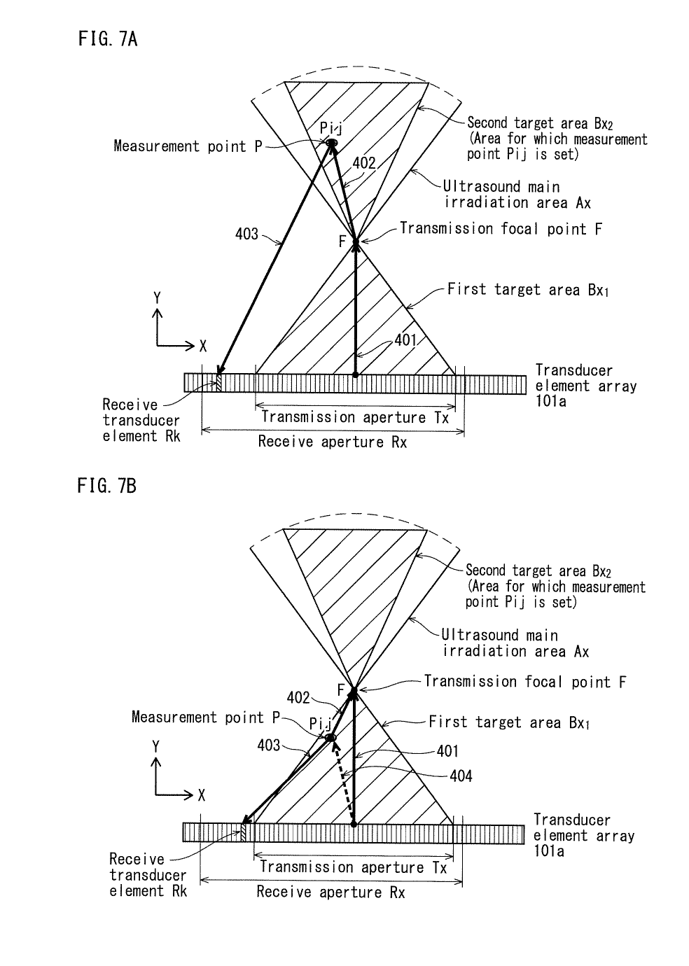

Each of FIGS. 7A and 7B is a schematic illustrating a propagation path of ultrasound that is transmitted from the transmission aperture Tx for a transmission event, is then reflected at a measurement point Pij of the target area Bx for the transmission event, and finally arrives at a receive transducer element Rk of the receive aperture Rx. Specifically, FIG. 7A illustrates the propagation path of ultrasound for a measurement point Pij located in the second target area Bx₂, that is, located deeper than the transmission focal depth. Meanwhile, FIG. 7B illustrates the propagation path of ultrasound for a measurement point Pij located in the first target area Bx₁, that is, located at or shallower than the transmission focal depth.

Following emission of ultrasound from the transmission aperture Tx, the wavefront of ultrasound converges at the transmission focal point F after proceeding along the path 401. Subsequently, the wavefront spreads out once again and arrives at the measurement point Pij. When there is a change in acoustic impedance at the measurement point Pij, transmitted ultrasound generates ultrasound reflection, which is received by the receive transducer elements Rk of the receive aperture Rx. The transmission focal point F is preset in advance upon designing of the transmission beam former 103. Thus, the length of the path 402 from the transmission focal point F to the measurement point Pij can be calculated geometrically.

The following describes how the transmission time is calculated in further detail.

First, the calculation of a transmission time for a measurement point Pij located in the second target area Bx₂ is described, with reference to FIG. 7A. A transmission time for a measurement point Pij located in the second target area Bx₂, is calculated assuming that ultrasound transmitted from the transmission aperture Tx arrives at the transmission focal point F by traveling along path 401, and then arrives at the measurement point Pij by traveling along path 402 from the transmission focal point F. As such, the transmission time for such a measurement point Pij is the total of the time amount required for transmitted ultrasound to travel through path 401 and the time amount required for transmitted ultrasound to travel through path 402. Specifically, the transmission time for such a measurement point Pij can be calculated, for example, by dividing the total of the lengths of paths 401 and 402 by the velocity at which ultrasound propagates within the subject.

In the meantime, the following describes the calculation of a transmission time for a measurement point Pij located in the first target area Bx₁, with reference to FIG. 7B. A transmission time for a measurement point Pij located in the first target area Bx₁ is calculated assuming that the time amount required for ultrasound transmitted from the transmission aperture Tx to arrive at the transmission focal point F by travelling along path 401 equals the time amount required for ultrasound transmitted from the transmission aperture Tx to travel along path 404 to arrive at the measurement point Pij and then travel along path 402 to arrive at the transmission focal point F from the measurement point Pij. As such, the transmission time for such a measurement point Pij is calculated by subtracting the time amount required for transmitted ultrasound to travel through the path 402 from the time amount required for transmitted ultrasound to travel through the path 401. Specifically, a transmission time for such a measurement point Pij can be calculated, for example, by dividing the value acquired by subtracting the length of path 401 from the length of path 401, by the velocity at which ultrasound propagates within the subject.

Note that in one or more embodiments, a transmission time for a measurement point Pij being located on the focal point is calculated, on the assumption that the measurement point Pij is located in the first target area Bx₁, by using a value obtained by subtracting the time amount required for transmitted ultrasound to travel through the path 402 from the time amount required for transmitted ultrasound to travel through the path 401. Alternatively, a transmission time for a measurement point Pij located on the focal point may be calculated, on the assumption that the measurement point Pij is located in the second target area Bx₂, by using the total of the time amount required for transmitted ultrasound to travel through path 401 and the time amount required for transmitted ultrasound to travel through path 402. This is because the length of the path 402 is zero in this case, and thus, the transmission time for a measurement point Pij located on the focal point equals the time amount required for transmitted ultrasound to travel through path 401 with either calculation method.

For each transmission event, the transmission time calculator 1044 calculates the transmission time for each measurement point Pij of the target area Bx for the transmission event. That is, the transmission time calculator 1044 calculates, for each measurement point Pij, the time amount required for transmitted ultrasound to arrive at the measurement point Pij. Further, the transmission time calculator 1044 outputs the transmission time so calculated to the delay amount calculator 1046.

### iv) Receive Time Calculator 1045

The receive time calculator 1045 is a circuit that calculates, for each measurement point P, a receive time required for ultrasound reflection from the measurement point P to arrive at each receive transducer element Rk of the receive aperture Rx. For a given transmission event, the receive time calculator 1045 acquires information indicating the positions of the receive transducer elements Rk for the given transmission event from the data storage 107, and acquires the information indicating the position of the target area Bx for the given transmission event from the target area setter 1042. Based on such information, the receive time calculator 1045, for each measurement point Pij of the target area Bx, calculates the receive time required for transmitted ultrasound to arrive at each receive transducer element Rk after being reflected at the measurement point Pij.

As already discussed above, transmitted ultrasound arriving at a measurement point Pij generates ultrasound reflection when there is a change in acoustic impedance at the measurement point Pij. The reflected ultrasound is then received by receive transducer elements Rk of the receive aperture Rx. As discussed above, the receive time calculator 1045 acquires information indicating the positions of the receive transducer elements Rk of the receive aperture Rx from the data storage 107. Accordingly, the receive time calculator 1045 is able to geometrically calculate the length of paths 403 leading from the measurement point Pij to the respective receive transducer elements Rk.

For each transmission event, the receive time calculator 1045 calculates the receive time for each measurement point Pij of the target area Bx for the transmission event. That is, the receive time calculator 1045 calculates, for each measurement point Pij, the time required for transmitted ultrasound to arrive at each receive transducer element Rk after being reflected at the measurement point Pij. Further, the receive time calculator 1045 outputs the receive time so calculated to the delay amount calculator 1046.

### v) Delay Amount Calculator 1046

The delay amount calculator 1046 is a circuit that calculates, for each receive transducer element Rk, a total propagation time based on the transmission time and the receive time for the receive transducer element Rk. Further, the delay amount calculator 1046 calculates, for each receive transducer element Rk, a delay amount to be applied to a receive signal sequence for the receive transducer element Rk. In specific, the delay amount calculator 1046 acquires, from the transmission time calculator 1044, the transmission time required for ultrasound waves to arrive at a measurement point Pij. Further, for each receive transducer element Rk, the delay amount calculator 1046 acquires, from the receive time calculator 1045, the receive time required for ultrasound to be reflected at the measurement point Pij and arrive at the receive transducer element Rk. Then, the delay amount calculator 1046, for each receive transducer Rk, calculates a total propagation time required for transmitted ultrasound to arrive at the receive transducer element Rk. Further, based on the difference between total propagation times for the receive transducer elements Rk, the delay amount calculator 1046 calculates a delay amount for each receive transducer element Rk. For each measurement point P of the target area Bx, the delay amount calculator 1046 calculates, for each receive transducer element Rk, the delay amount to be applied to a receive signal sequence for the receive transducer element Rk, and outputs the delay amounts to the delay processor 1047.

### vi) Delay Processor 1047

The delay processor 1047 is a circuit that specifies, for each receive transducer element Rk, a receive signal based on reflected ultrasound from a measurement point Pij. In specific, for each receive transducer element Rk, the delay processor 1047 specifies a receive signal corresponding to the delay amount for the receive transducer element Rk from the receive signal sequence for the receive transducer element Rk.

More specifically, for each transmission event, the delay processor 1047 acquires, for each receive transducer element Rk, information indicating the position of the receive transducer element Rk from the receive aperture setter 1043, the receive signal sequence for the receive transducer element Rk from the data storage 107, and the delay amount to be applied to the receive signal sequence of the receive transducer element Rk from the delay amount calculator 1046. In addition, for each transmission event, the delay processor 1047 acquires the information indicating the position of the target area Bx from the target area setter 1042. Further, for each receive transducer element Rk, the delay processor 1047 specifies a receive signal based on reflected ultrasound from a measurement point Pij. In specific, the delay processor 1047 specifies, from the receive signal sequence for the receive transducer element Rk, a receive signal corresponding to a time point after subtraction of the delay amount for the receive transducer element Rk. The delay processor 1047 outputs the receive signal so specified to the sum calculator 1049.

### vii) Weight Calculator 1048

The weight calculator 1048 is a circuit that calculates a weight sequence (reception apodization weight) for the receive transducer elements Rk, so that the maximum weight is set with respect to the receive transducer element located at the center of the receive aperture Rx in the transducer element array direction.

As illustrated in FIG. 6, the weight sequence is a numerical sequence of weight coefficients that are to be applied to receive signals for the receive transducer elements composing the receive aperture Rx. The weight sequence indicates weights that are distributed symmetrically with respect to the measurement point Pij. As the shape of distribution of the weights indicated by the weight sequence, any shape is applicable, including but not limited to a hamming window, a hanning window, and a rectangular window. The weight sequence is set so that the maximum weight is set with respect to the receive transducer element located at the center position of the receive aperture Rx in the transducer element array direction, and the central axis of the weight distribution corresponds to the central axis Rxo of the receive aperture Rx. The weight calculator 1048 uses as input information indicating the positions of the receive transducer elements Rk, which is output from the receive aperture setter 1043, and outputs the weight sequence for the receive transducer elements Rk to the sum calculator 1049.

### viii) Sum Calculator 1049

The sum calculator 1049 is a circuit that generates a delayed-and-summed acoustic line signal for each measurement point P, by using as input the specified receive signals for the receive transducer elements Rk, which are output from the delay processor 1047, and summing together the specified receive signals. Alternatively, the sum calculator 1049 may generate an acoustic line signal for each measurement point P by using as input the weight numerical sequence for the receive transducer elements Rk, which is output from the weight calculator 1048, multiplying the specified receive signal for each receive transducer element Rk with a corresponding weight, and summing the weighted receive signals. The sum calculator 1049 sums the receive signals for the receive transducer elements Rk, after the receive signals have been put in the same phase by the delay processor 1047. Due to this, the sum calculator 1049 is capable of increasing the S/N ratio of the receive signals received by the receive transducer elements Rk based on reflected ultrasound from the measurement point Pij, and receive signals for the measurement point Pij can be extracted.

As a result of one transmission event and processing accompanying the transmission event, an acoustic line signal is generated for each measurement point P of the target area Bx for the transmission event. Further, by repetitively performing transmission events while shifting the transmission aperture Tx in the transducer element array direction by the shift pitch Mp each time, all of the transducer elements 101a in the probe 101 perform ultrasound transmission. Due to this, a frame acoustic line signal, which is a synthesizing result of acoustic line signals corresponding to one frame, is generated.

In one or more embodiments, acoustic line signals for respective measurement points, which compose the frame acoustic line signal and each of which is generated by synthesizing a plurality of acoustic lines signals corresponding to the measurement point that are included in different sub-frame acoustic line signals, are each referred to as a synthesized acoustic line signal for the measurement point.

The sum calculator 1049, for each transmission event, generates a sub-frame acoustic line signal as a synthesizing result of acoustic line signals for every measurement point Pij of the target area Bx for the transmission event. Further, the sum calculator 1049 outputs the sub-frame acoustic line signals so generated to be stored in the data storage 107.

### (5) Synthesizer 1140

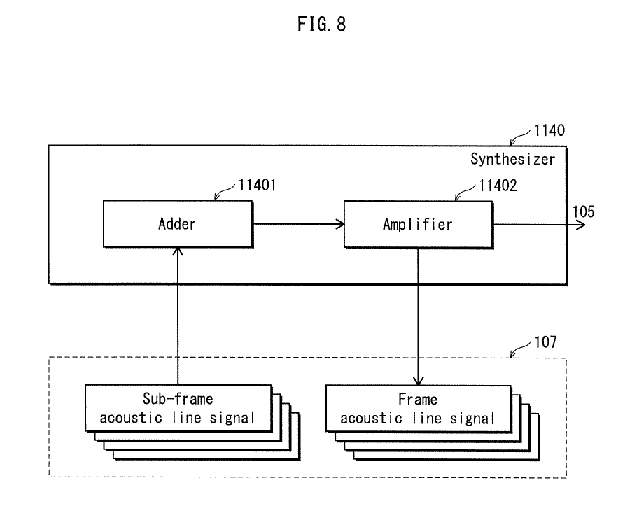

The synthesizer 1140 is a circuit that generates a frame acoustic line signal by synthesizing a plurality of sub-frame acoustic line signals each generated for one transmission event. FIG. 8 is a functional block diagram illustrating the structure of the synthesizer 1140. As illustrated in FIG. 8, the synthesizer 1140 includes an adder 11401 and an amplifier 11402.

The following describes the structure of each functional block of the synthesizer 1140.

### i) Adder 11401

The adder 11401, after the generation of a series of sub-frame acoustic line signals necessary for generating one frame acoustic line signal is completed, reads out the sub-frame acoustic line signals from the data storage 107. Further, the adder 11401 generates a frame acoustic line signal by synthesizing the plurality of sub-frame acoustic line signals. The synthesizing of the sub-frame acoustic line signals is performed according to the positions of the measurement points Pij, such that in the process, a synthesized acoustic line signal is generated for each measurement point Pij. In specific, the adder 11401 generates a synthesized acoustic line signal for a given measurement point Pij by synthesizing a plurality of acoustic line signals corresponding to the measurement point Pij that are included in different sub-frame acoustic line signals. Due to this, acoustic line signals for the same measurement point that are included in different sub-frame acoustic line signals are synthesized, to generate a synthesized acoustic line signal for the measurement point.

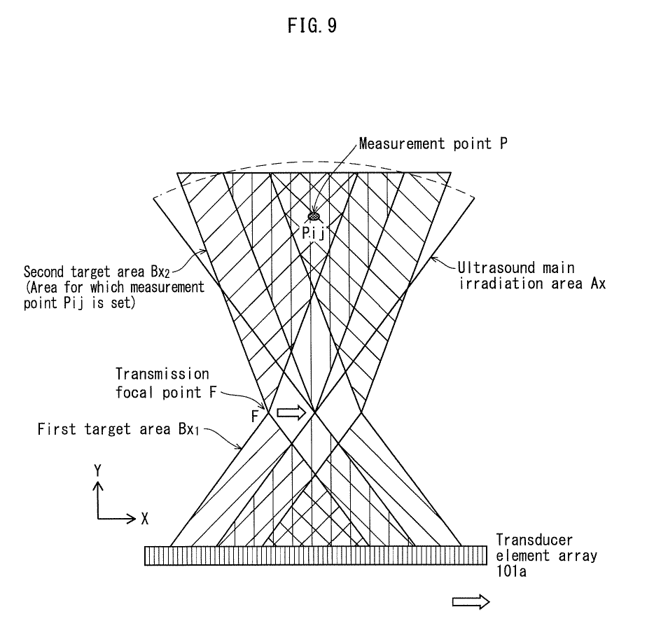

FIG. 9 is a schematic illustrating processing by the adder 11401 for generating a synthesized acoustic line signal. As already discussed above, ultrasound transmission is performed by repetitively performing transmission events while shifting the transmission transducer element array (i.e., the transmission aperture Tx) in the transducer element array direction each time. Due to this, target areas Bx for two consecutive transmission events differ in position from one another in the transducer element array direction by a width of a single transducer element. Thus, a frame acoustic line signal covering all target areas Bx can be generated by synthesizing sub-frame acoustic line signals based on the positions of the measurement points Pij from which the acoustic lines signals included in the sub-frame acoustic line signals are acquired.

Further, for a measurement point included in multiple target areas Bx, values of a plurality of acoustic line signals included in different sub-frame acoustic line signals are summed. Thus, the synthesized acoustic line signal for such a measurement point may indicate a great value, depending upon the number of target areas Bx in which the measurement point is included. In the following, the number of different target areas Bx in which a given measurement point is included is referred to as an overlap count of the measurement point, and the maximum value of the overlap count in the transducer element array direction is referred to as a maximum overlap count.

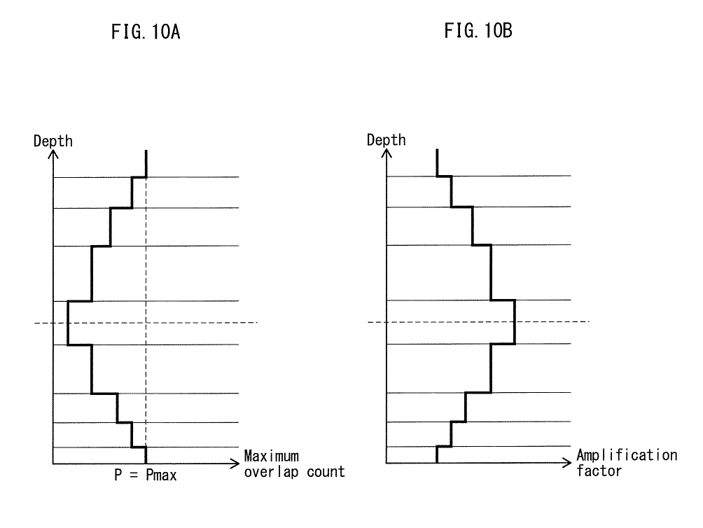

Further, in one or more embodiments, the target area Bx has an hourglass-shape. Due to this, the overlap count and the maximum overlap count fluctuate in the depth direction of the subject, as illustrated in FIG. 10A. Accordingly, there is a depth-direction fluctuation in values of synthesized acoustic line signals. Note that, as described above, the second target area Bx₂ has a less fluctuation of the width in the transducer element array direction relative to the distance from the focal point F than the first target area Bx₁. Due to this, the second target area Bx₂ has a less fluctuation of the overlap count relative to the depth than the first target area Bx₁.

Note that in synthesizing sub-frame acoustic line signals based on the positions of the measurement points Pij from which the acoustic lines signals included in the sub-frame acoustic line signals are acquired to generate synthesized acoustic line signals for the respective measurement points, the adder 11401 may add weights in accordance with the positions of the measurement points Pij.

The adder 11401 outputs the frame acoustic line signal so generated to the amplifier 10492.

### ii) Amplifier 11402

As already described above, there is a depth-direction fluctuation in values of synthesized acoustic line signals. In order to moderate such fluctuation in values of different synthesized acoustic line signals, the amplifier 11402, in synthesizing the synthesized acoustic line signals to generate the frame acoustic line signal, performs amplification of multiplying the synthesized acoustic line signals by amplification factors. Here, the amplifier 11402 determines an amplification factor for a given synthesized acoustic line signal according to the number of acoustic line signals synthesized to yield the synthesized acoustic line signal.

FIG. 10B is a schematic providing an overview of the amplification performed by the amplifier 11402. The maximum overlap count fluctuates in the depth direction, as illustrated in FIG. 10B. Thus, to compensate with this fluctuation in maximum overlap count, the amplifier 11402 multiplies the synthesized acoustic line signals by respective amplification factors that are based on the maximum overlap counts and vary in the depth direction, as illustrated in FIG. 10B. This moderates a difference between values of synthesized acoustic line signals deriving from the fluctuation in overlap counts in the depth direction, and thus, the values of the synthesized acoustic line signals after the amplification are averaged out in the depth direction. That is, the amplification performed by the amplifier 11402 is gain equalization in the depth direction.

Further, the amplifier 11402 may also multiply the synthesized acoustic line signals by amplification factors varying in the transducer element array direction that are calculated based on overlap counts, when overlap counts fluctuate in the transducer element array direction. This moderates a difference between values of synthesized acoustic line signals deriving from the fluctuation in overlap counts in the transducer element array direction, and thus, the values of the synthesized acoustic line signals after the amplification are averaged out in the transducer element array direction.

Here, note that the amplifier 11402 may generate the frame acoustic line signal by synthesizing amplified synthesized acoustic line signals for respective measurement points.

### Operations

The following describes the operations of the ultrasound diagnostic device 100 having the structure described up to this point.

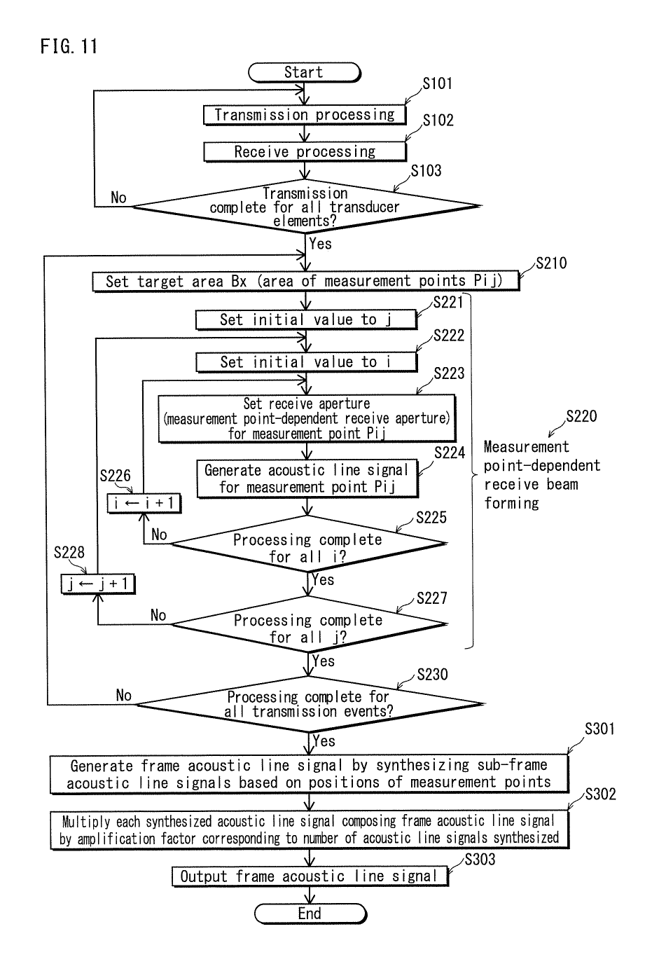

FIG. 11 is a flowchart illustrating beam forming by the receive beam former 104.

First, in Step S101, the transmitter 1031 performs transmission processing (a transmission event) of supplying a transmission signal causing transmission of an ultrasound beam to each transmission transducer element of the transmission aperture Tx.

In Step S102, the receiver 1040 generates receive signal sequences based on electric signals yielded through the reception of reflected ultrasound by the probe 101, and outputs the receive signal sequences to be stored in the data storage 107. Then, a determination is made of whether or not all transducer elements 101a of the probe 101 have performed ultrasound transmission (S103). When one or more of the transducer elements 101a have not yet performed ultrasound transmission, processing returns to Step S101, which results in another transmission event being executed by shifting the transmission aperture Tx in the transducer element array direction by the shift pitch Mp. Meanwhile, when all of the transducer elements 101a have performed ultrasound transmission, processing proceeds to Step S210.

In Step S210, the target area setter 1042 sets a target area Bx for a processing-target transmission event based on information indicating the position of the transmission aperture Tx for the processing-target transmission event. In the initial loop of processing, the target area setter 1042 sets a target area Bx for the initial transmission event, which can be calculated from the transmission aperture Tx for the initial transmission event.

Subsequently, processing proceeds to measurement-point dependent beam forming (Step S220 (including Steps S221 through S228)). In Step S220, first, coordinate values i and j indicating a position of a measurement point Pij of the target area Bx for the processing-target transmission event are initialized (set to the respective minimum possible values in the target area Bx) (Steps S221 and S222). Then, the receive aperture setter 1043 sets a receive aperture Rx for the current measurement point so that the center of the receive aperture Rx corresponds to a transducer element Xk that is spatially closest to the current measurement point Pij (Step S223).

Subsequently, an acoustic line signal is generated for the current measurement point Pij (Step S224).

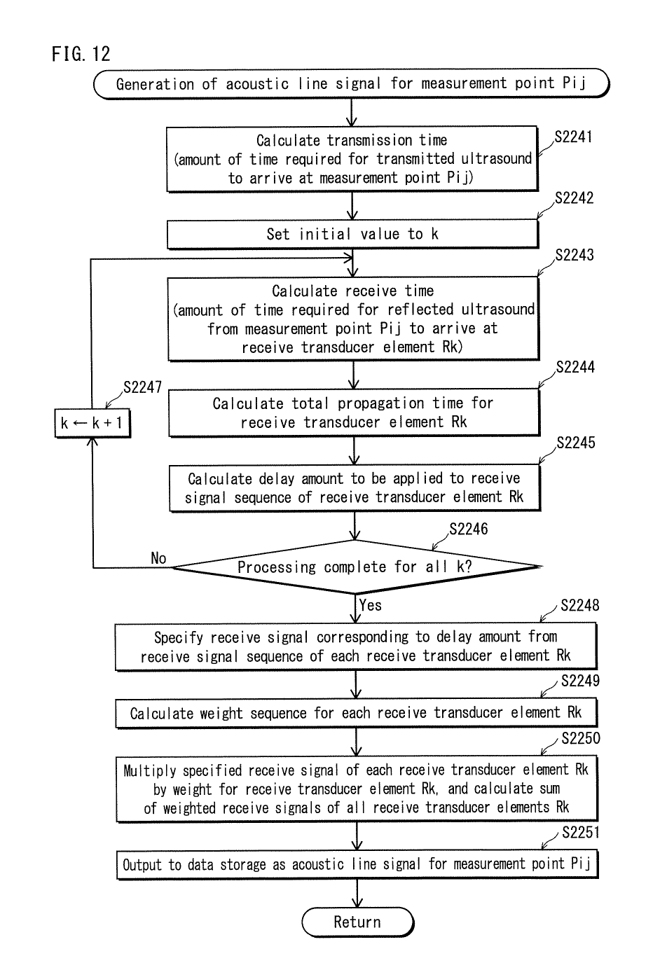

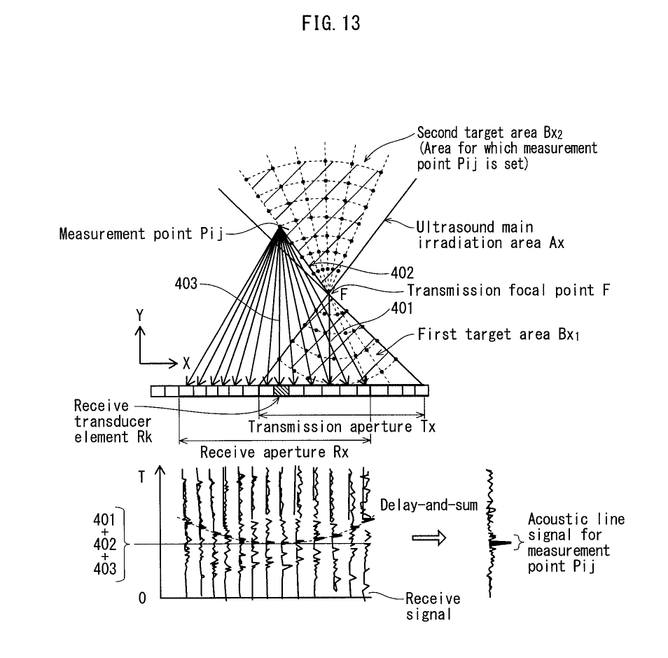

The following describes the operations in Step S224 for generating an acoustic line signal for the current measurement point Pij. FIG. 12 is a flowchart illustrating the operations of the receive beam former 104 for generating the acoustic line signal for the current measurement point Pij. FIG. 13 is a schematic for explaining the operations of the receive beam former 104 for generating the acoustic line signal for the current measurement point Pij.

First, in Step S2241, the transmission time calculator 1044 calculates, for the current measurement point Pij, a transmission time required for transmitted ultrasound to arrive at the current measurement point Pij. As already described above, the current measurement point Pij is a measurement point of the target area Bx for the processing-target transmission event. Here, (i) when the current measurement point Pij is located in the second target area Bx₂, the transmission time for the current measurement point Pij is calculated by dividing, by ultrasound velocity cs, the geometrically-calculable length of a path (combination of paths 401 and 402) starting at a transmission transducer element in the transmission aperture Tx and reaching the current measurement point Pij via the transmission focal point F. Meanwhile, (ii) when the current measurement point Pij is located in the first target area Bx₁, the transmission time for the current measurement point is calculated by dividing, by the ultrasound velocity cs, a value (401-402) obtained by subtracting the geometrically-calculable length of the path from the transmission focal point F to the current measurement point Pij from the geometrically-calculable length of the path from a transmission transducer element in the transmission aperture Tx to the transmission focal point F.

Subsequently, value k, which indicates the position of a target receive transducer element Rk of the receive aperture Rx, is initialized (set to the minimum possible value in the receive aperture Rx) (Step S2242). Then, the receive time for the target receive transducer element Rk is calculated (Step S2243). The receive time is the time required for transmitted ultrasound to arrive at the target receive transducer element Rk after being reflected at the current measurement point Pij. The receive time for the target receive transducer element Rk can be calculated by dividing, by the ultrasound velocity cs, the geometrically-calculable length of the path 403 from the current measurement point Pij to the target receive transducer element Rk. Further, from a sum of the transmission time and the receive time for the target receive transducer element Rk, the total propagation time required for ultrasound transmitted from the transmission aperture Tx to arrive at the target receive transducer element Rk after being reflected at the current measurement point Pij is calculated (Step S2244). Further, based on the difference in total propagation time between different receive transducer elements Rk composing the receive aperture Rx, the delay amount for the target receive transducer element Rk is calculated (Step S2245).

Subsequently, a determination is performed of whether or not a delay amount has been calculated for every receive transducer element Rk composing the receive aperture Rx (Step S2246). When a delay amount has not yet been calculated for one or more of the receive transducer elements Rk, the value k is incremented (Step S2247), and a delay amount for another receive transducer element Rk is calculated (Step S2243). Meanwhile, when a delay amount has been calculated for every receive transducer element Rk composing the receive aperture Rx, processing proceeds to Step S2248. Note that at this point, a delay amount for the current measurement point Pij has already been calculated for each receive transducer element Rk of the receive aperture Rx. The delay amount for a given receive transducer element Rk indicates delay with which reflected ultrasound from the current measurement point Pij arrives at the receive transducer element Rk.

In Step S2248, the delay processor 1047, for each receive transducer element Rk, specifies a receive signal based on reflected ultrasound from the current measurement point Pij. Here, the delay processor 1047 specifies, from a receive signal sequence corresponding to each receive transducer element Rk, a receive signal corresponding to a time point after subtraction of the delay amount for the receive transducer element Rk.

Subsequently, the weight calculator 1048 calculates a weight sequence for the receive transducer elements Rk of the current receive aperture Rx, so that the maximum weight is set with respect to the receive transducer element located at the center position of the receive aperture Rx in the transducer element array direction (S2249). Then, the sum calculator 1049 generates an acoustic line signal for the current measurement point Pij by multiplying the specified receive signal for each receive transducer element Rk by a weight corresponding to the receive transducer element Rk, and summing the weighted receive signals for the different receive transducer elements Rk (Step S2250). Following this, the sum calculator 1049 outputs the acoustic line signal for the current measurement point Pij to the data storage 107 to be stored in the data storage 107 (Step S2251).

Referring to FIG. 11 once again, subsequently, an acoustic line signal is generated for each measurement point Pij (each illustrated in FIG. 13 as a black dot) of the target area Bx for the processing-target transmission event, by repeating Steps S223, S224 while incrementing the coordinate values i and j (Steps S225, S227). Subsequently, a determination is performed of whether or not an acoustic line signal has been generated for every measurement point Pij of the target area Bx. When an acoustic line signal has not yet been generated for every measurement point Pij of the target area Bx, the coordinate values i and j are incremented, yielding an acoustic line signal for another measurement point Pij (Step S224). Meanwhile, when an acoustic line signal has already been generated for every measurement point Pij of the target area Bx, processing proceeds to Step S230. At this point, an acoustic line signal has already been generated for each measurement point P of the target area Bx corresponding to the processing-target transmission event, and the acoustic line signals have been output to and stored to the data storage 107. In other words, a sub-frame acoustic line signal for the processing-target transmission event has been generated, and output to and stored to the data storage 107.

Subsequently, a determination is performed of whether or not a sub-frame acoustic line signal has been generated for each transmission event having been performed (Step S230). When sub-frame acoustic line signals have not yet been generated for one or more transmission events, processing proceeds to Step S210, where the coordinate values i and j are initialized (set to the respective minimum possible values in the target area Bx for the subsequent transmission event, which can be calculated from the transmission aperture Tx for the subsequent transmission event) (Steps S221 and S222), and then setting of a receive aperture Rx is performed (Step S223) and generation of acoustic line signals (Step S224). Meanwhile, when sub-frame acoustic line signals have been generated for every transmission event having been performed, processing proceeds to Step S301.

In Step S301, the adder 11401 reads out the sub-frame acoustic line signals stored in the data storage 107, and combines the sub-frame acoustic line signals based on positions of the measurement points Pij. Thus, a synthesized acoustic line signal is generated for each measurement point Pij, and accordingly, a frame acoustic line signal is generated. Subsequently, the amplifier 11402 multiples each synthesized acoustic line signal by a corresponding amplification factor that is determined based on the number of acoustic line signals, included in the sub-frame acoustic line signals, that have been synthesized to yield the synthesized acoustic line signal (Step S302). Further, the amplifier 11402 outputs the amplified frame acoustic line signal to the ultrasound image generator 105 and the data storage 107 (Step S303), and processing is terminated.

### Conclusion

As described above, the ultrasound diagnostic device 100 pertaining to one or more embodiments, according to the synthetic aperture method, synthesizes acoustic line signals for the same measurement point that are generated from different transmission events. This achieves the effect of performing, for multiple transmission events, virtual transmission focusing even for measurement points that are located in depths other than that of the transmission focal point F. This improves spatial resolution and S/N ratio.

Also, in the ultrasound diagnostic device 100, with respect to the target area included in the ultrasound main irradiation area for which sub-frame acoustic line signals are to be generated, the first target area is set so as to include the entirety of an area located at or shallower than the focal depth. Accordingly, with respect to shallower areas for which both spatial resolution and S/N ratio are expected to be high, it is possible to improve the use efficiency of ultrasound and enjoy the effect of the synthetic aperture method of improving spatial resolution and S/N ratio to the maximum. Meanwhile, in an area located deeper than the focal depth, the second target area is set so as to have a smaller increase in width in the transducer element array direction as departing the focal point than the first target area has. Accordingly, it is possible to reduce the number of measurement points especially at deep areas for which S/N ratio is not sufficiently improved even by delay-and-summing. Moreover, the second target area Bx₂, has the central axis that coincides the central axis of the ultrasound main irradiation area Ax. Amplitude of transmitted ultrasound beam is not necessarily constant in the entire ultrasound main irradiation area Ax, and decreases as departing the central axis of the ultrasound main irradiation area Ax. Also, sensitivity of reception transducer elements for reflected ultrasound from a measurement point decreases as departing the central axis of the ultrasound main irradiation area. Accordingly, by setting the second target area Bx₂ so as to include an area close to the central axis of the ultrasound main irradiation area, it is possible to set the second target area Bx₂, which is located deeper than the focal depth, so as to include measurements points for which S/N ratio is high and exclude measurements points for which S/N ratio is low. This allows a considerable reduction in delay-and-summing computation amount while minimalizing the influence of degradation of frame acoustic line signal quality.

Further, in the ultrasound diagnostic device 100, the receive aperture setter 1043 selects, as transducer elements composing the receive aperture Rx for each measurement point P, transducer elements forming an array whose center position in the transducer element array direction matches a transducer element that is spatially closest to the measurement point P. Accordingly, the ultrasound diagnostic device 100 performs receive beam forming by using a receive aperture that is not dependent upon ultrasound transmission events but is dependent upon the position of the measurement point P, and that is symmetric with respect to the measurement point P. Due to this, the receive aperture Rx for a given measurement point P does not change (i.e., the same receive aperture Rx is used for the same measurement point P) between different transmission events, between which the transmission focal point F is shifted in the transducer element array direction. Thus, delay-and-sum processing for the same measurement point P is always performed by using the same receive aperture Rx. In addition, in the ultrasound diagnostic device 100, a weight sequence is set so that the closer a receive transducer element is to the measurement point P, the greater the weight applied to the receive transducer element. Due to this, even taking into account the fact that ultrasound decay increases as propagation distance increases, ultrasound reflected from the measurement point P can be used with high efficiency. Accordingly, the ultrasound diagnostic device 100 achieves both high local spatial resolution and high S/N ratio.

### Modification 1 of One or More Embodiments

The receive aperture setter 1043 in the ultrasound diagnostic device 100 pertaining to one or more embodiments sets, for each measurement point P, the receive aperture Rx so that the center position of the receive aperture Rx in the transducer element array direction corresponds to a transducer element that is spatially closest to the measurement point P. However, the configuration of the receive aperture Rx may be changed as necessary, as long as acoustic line signals for all measurement points Pij of the target area Bx can be generated by calculating total propagation times and performing delaying based on total propagation paths. As already discussed above, a total propagation time for a given receive transducer element Rk is the time required for ultrasound transmitted from the transmission aperture Tx to reach the receive transducer element Rk after passing through the transmission focal point F and being reflected at the measurement point P.

Modification 1 provides an ultrasound diagnostic device differing from the ultrasound diagnostic device 100 pertaining to one or more embodiments for including a receive aperture setter (a Tx receive aperture setter) that sets, for each transmission event, the receive aperture Rx so that the center position of the receive aperture Rx corresponds to the center position of the transmission aperture Tx for the transmission event. That is, the receive aperture Rx in modification 1 can be referred to as a transmission-dependent receive aperture. Other than the Tx receive aperture setter, the components of the ultrasound diagnostic device pertaining to modification 1 have the same structures and configurations as the corresponding components in the ultrasound diagnostic device 100 described in one or more embodiments. Thus, description of such similar components is not provided in the following.

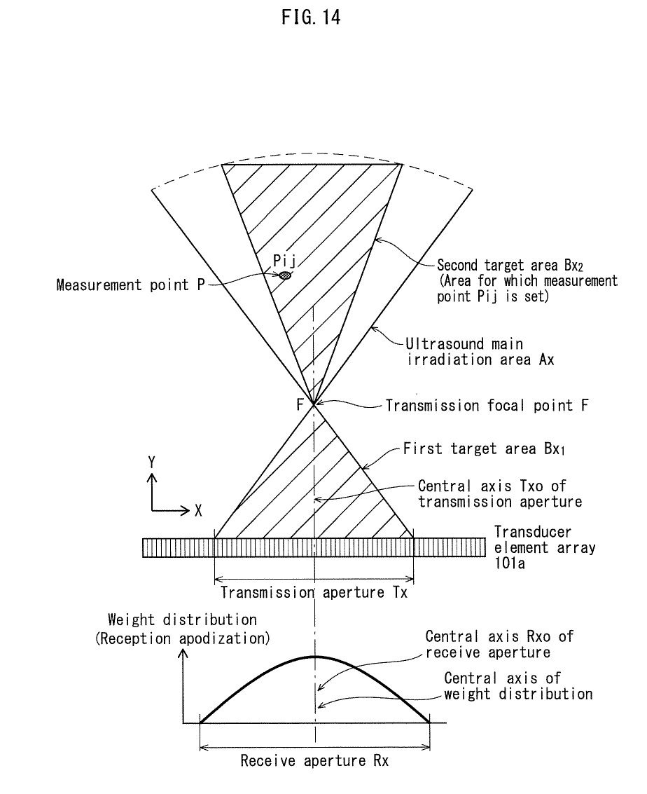

FIG. 14 is a schematic illustrating the relationship between a transmission aperture Tx and a receive aperture Rx set by the Tx receive aperture setter. In modification 1, the Tx receive aperture setter sets, for each transmission event, a receive aperture Rx so that the center position of the receive aperture Rx in the transmission element array direction corresponds to the center position of the transmission aperture Tx for the transmission event. Thus, the position of an axis Rxo passing through the center position of the receive aperture Rx corresponds to the position of an axis Txo passing through the center position of the transmission aperture Tx. Further, the receive aperture Rx is symmetric about the transmission focal point F (i.e., has the same number of apertures at both sides of the center position thereof in the transmission element array direction). As such, as the transmission aperture Tx shifts in the transducer element array direction from one transmission event to another, the receive aperture Rx also shifts in the transducer element array direction, following the transmission aperture Tx.

In addition, a weight sequence (so-called reception apodization weight) for the receive transducer elements Rk is calculated, so that the maximum weight is set with respect to the receive transducer element Rk located along the central axis Rxo of the receive aperture Rx and the central axis Txo of the transmission aperture Tx. The weight sequence indicates weights distributed symmetrically with respect to the transducer element Xk. As the shape of distribution of the weights indicated by the weight sequence, any shape is applicable, including but not limited to a hamming window, a hanning window, and a rectangular window.

### Operations

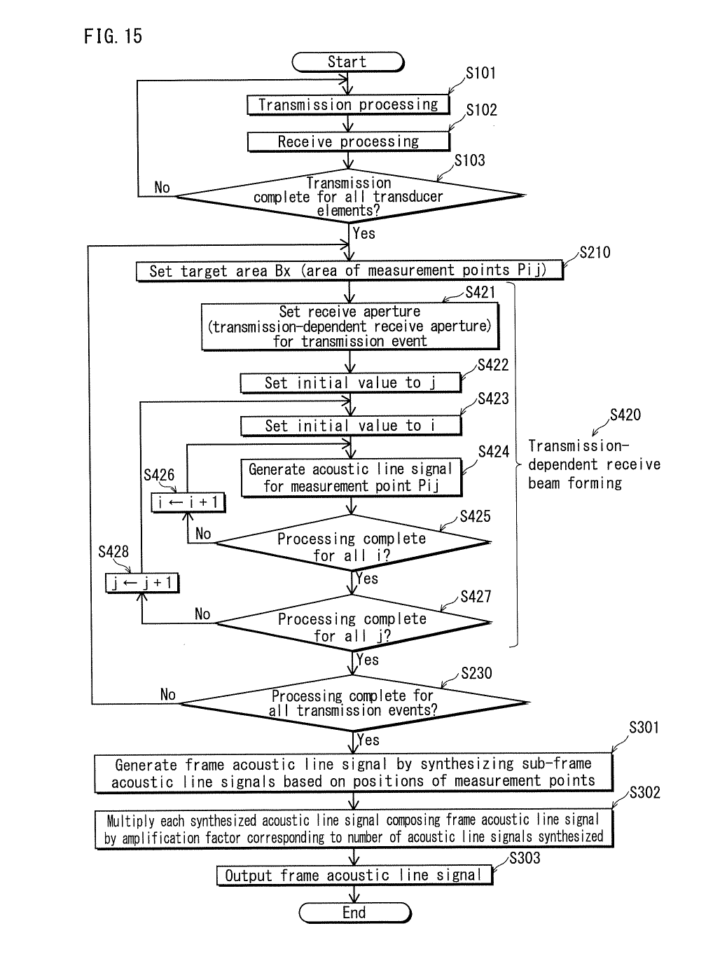

FIG. 15 is a flowchart illustrating beam forming by a receive beam former of the ultrasound diagnostic device pertaining to modification 1. The flowchart in FIG. 15 differs from the flowchart in FIG. 11 because transmission-dependent receive beam forming (Step S420 (including Steps S421 through S428)) is performed in place of measurement point-dependent beam forming (Step S220 (including Steps S221 through S228)), respectively. Meanwhile, the processing in steps other than Step S420 in the flowchart in FIG. 11 is similar to the processing in the corresponding steps in the flowchart in FIG. 11. Thus, description of such similar processing is not provided in the following.

In Step S420, first, the Tx receive aperture setter sets a receive aperture Rx for a transmission event by selecting receive transducer elements Rk composing a receive transducer element array whose center position matches the center position of the transducer element array composing the transmission aperture Tx for the corresponding transmission event, in Step S421.

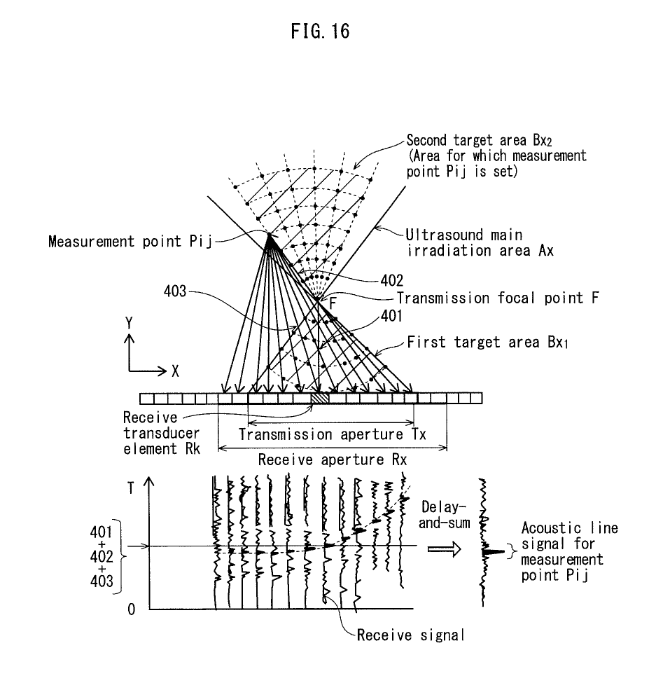

Subsequently, coordinate values i and j indicating a position of a measurement point Pij of the target area Bx for the processing-target transmission event are initialized (set to the respective minimum possible values in the target area Bx set in Step S210) (Steps S422 and S423). Subsequently, an acoustic line signal is generated for the current measurement point Pij (Step S424). FIG. 16 is a schematic for explaining the operations of the receive beam former pertaining to modification 1 for generating the acoustic line signal for the current measurement point Pij. FIG. 16 differs from FIG. 13 referred to in one or more embodiments in terms of the positional relationship between the transmission aperture Tx and the receive aperture Rx. The processing in Step S424 is similar to that in Step S224 of FIG. 11 (i.e., Steps S2241 through S2251 in FIG. 12).

An acoustic line signal is generated for each measurement point Pij (each illustrated in FIG. 16 as a black dot) of the target area Bx by repeating Step S424 while incrementing the coordinate values i and j. Subsequently, a determination is performed of whether an acoustic line signal has not yet been generated for one or more of the measurement points Pij of the target area Bx (Steps S425, S427). When an acoustic line signal has not yet been generated for every measurement point Pij of the target area Bx, the coordinate values i and j are incremented (Steps S426 and S428), yielding an acoustic line signal for another measurement point Pij (Step S424). Meanwhile, when an acoustic line signal has already been generated for every measurement point Pij of the target area Bx, processing proceeds to Step S230. At this point, an acoustic line signal has already been generated for each measurement point Pij of the target area Bx for the processing-target transmission event, and the acoustic line signals have been output to and stored to the data storage 107.

### Effects

The ultrasound diagnostic device pertaining to modification 1, which has been described up to this point, achieves the effects described in one or more embodiments, excluding the effect related to setting a measurement point-dependent receive aperture. In place of the effect related to setting a measurement point-dependent receive aperture, the ultrasound diagnostic device pertaining to modification 1 achieves the following effect. In modification 1, for each transmission event, the receive aperture Rx is set by selecting receive transducer elements forming a transducer element array whose center position corresponds to the center position of the transducer element array composing the transmission aperture Tx for the transmission event. Due to this, the position of the central axis Rxo of the receive aperture Rx for a given transmission event corresponds to the position of the central axis Txo of the transmission aperture Tx for the same transmission event. Further, when transmission events are repetitively performed, the transmission aperture Tx shifts in the transducer element array direction each time, and the receive aperture Rx also shifts in the transducer element array direction in synchronization with the transmission aperture Tx. Thus, a different receive aperture is used to perform delay-and-sum for each transmission event. Accordingly, receive processing with respect to multiple transmission events can be performed by using a group of receive apertures covering a vast measurement area and each differing in terms of time. Thus, uniform spatial resolution is achieved over a vast measurement area.

### Modification 2 of One or More Embodiments

In the ultrasound diagnostic devices pertaining to one or more embodiments, the shape of the second target area Bx₂ is set by decreasing the width of the similar shape of the first target area Bx₁ in the transducer element array direction by n times (1>n>0). Alternatively, the second target area Bx₂ may be in the following shape.

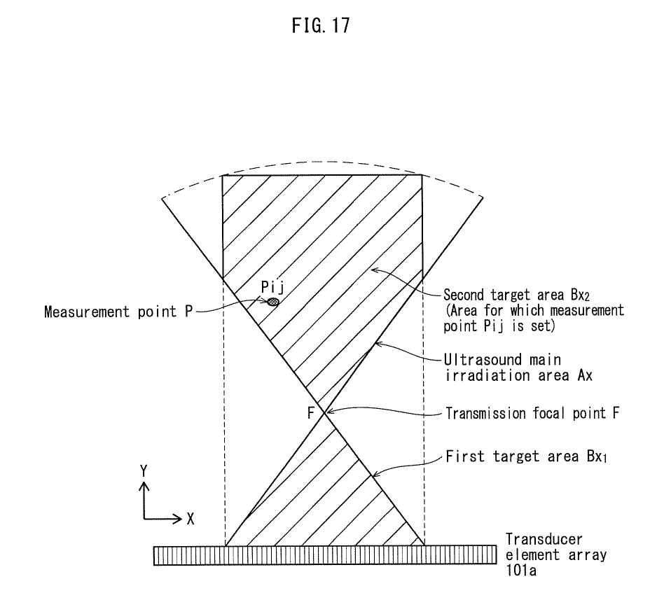

FIG. 17 illustrates a first setting example of the second target area Bx₂ pertaining to modification 2. As illustrated in FIG. 17, the second target area Bx₂ is set to be, at areas deeper than the focal depth in the ultrasound main irradiation area Ax, an inner part of a rectangle having the transmission aperture Tx as a bottom. Thus, when the focal depth is represented by Df, the second target area Bx₂ has the identical shape with the first target area Bx₁ at a range from Df to 2×Df. Meanwhile, at a depth greater than 2×Df, the second target area Bx₂ is a stripe-shaped area whose width in the transducer element array direction is equal to the width of the transmission aperture Tx. Specifically, the second target area Bx₂ is a pentagon that is formed by connecting a triangle, which is identical with the first target area Bx₁, with a rectangle that has a bottom of the triangle as one of sides. By setting the second target area Bx₂, in this way, it is possible to, in areas at a range from Df to 2×Df as well as areas at or shallower than Df, improve the use efficiency of ultrasound and enjoy the effect of the synthetic aperture method of improving spatial resolution and S/N ratio to the maximum. Meanwhile, in areas deeper than 2×Df, its width is constant irrespective of the maximum depth of the second target area Bx₂, and accordingly the number of measurement points does not greatly increase. Thus, especially in the case where the focal depth is small relative to the maximum depth of the second target area Bx₂ (that is, the focal depth is shallow relative to an ROI), it is possible to suppress an increase in computation amount while improving spatial resolution and S/N ratio of acoustic line signals at depths twice the focal depth. Note that the following configuration may be employed that in the case where the maximum width of the first target area Bx₁ in the transducer element array direction is smaller than the width of the transmission aperture Tx, the maximum width of the second target area Bx₂ is smaller than the maximum width of the first target area Bx₁. With this configuration, it is possible to further reduce the number of measurement points located in the second target area Bx₂.

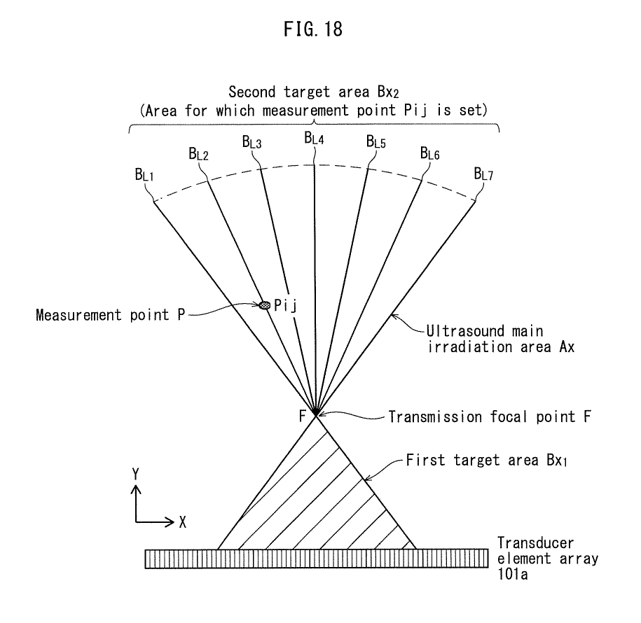

FIG. 18 illustrates a second setting example of the second target area Bx₂ pertaining to modification 2. As illustrated in FIG. 18, the second target area Bx₂ is composed of a plurality of target lines B_{L1}-B_{L7} each of which is located on an outer boundary of the ultrasound main irradiation area Ax or is located inside the ultrasound main irradiation area Ax. The target lines are each a half line starting from the focal point F or the focal area. The target lines B_{L1} and B_{L7} each correspond to the outer boundary of the ultrasound main irradiation area Ax, and the target line B_{L4} is located on the central axis Txo of the transmission aperture Tx. For the sake of convenience, the following description is provided based on the assumption that the ultrasound main irradiation area Ax has two outer boundaries, one being a straight line passing through the focal point F and one end of the transmission aperture Tx, and the other being a straight line passing through the focal point F and the other end of the transmission aperture Tx. In other words, the second target area Bx₂ has a measurement point density in the transducer element array direction that is at least one-half, one-fourth, or one-eighth of the measurement point density in the depth direction. This configuration allows arranging measurement points uniformly over substantially the entire area deeper than the focal depth in the ultrasound main irradiation area Ax, such that the measurement point density is high in the depth direction and low in the transducer element array direction. Thus, the number of measurement points located in the second target area Bx₂ decreases in proportion to the measurement point density in the transducer element array direction. According to the second setting example, in the case where the number of measurement points located in the second target area Bx₂ is nearly equal to that in the second target area Bx₂ pertaining to one or more embodiments, it is possible to improve spatial resolution and S/N ratio in areas deeper than the focal depth compared with in one or more embodiments. This is owing to the following two effects. (i) The travel direction of ultrasound beam varies in an increased range among multiple transmission events, and thus complementation is sufficiently performed by synthesizing acoustic line signals acquired through ultrasound beams with different travel directions. (ii) The positional relationship among the measurement point, the focal point F, and the receive aperture greatly varies among multiple transmission events, and thus S/N ratio is improved. Accordingly, it is possible to realize either point (i) or (ii): (i) improvement of spatial resolution and SIN ratio in the case where computation amount is reduced as much as that in one or more embodiments; and (ii) further reduction in computation amount in the case where spatial resolution and S/N ratio are achieved as much as that in one or more embodiments.

Note that the number of target lines is not limited to seven, and may be arbitrary. Also, the target lines may be located such that measurement points on the target lines are spaced away from one another at equal distance, or such that every pair of adjacent ones of the target lines forms a predetermined angle therebetween. Further, the target lines may be located such that the distance between measurement points in the transducer element array direction decreases as approaching the central axis Txo of the transmission aperture Tx, and increases as departing the central axis Txo. With this configuration, it is possible to eccentrically locate measurement points in areas deeper than the focal depth from which receive signals having a high S/N ratio are obtained. This allows weight addition in accordance with S/N ratio of receive signals while increasing the range of ultrasound beam travel directions and the range of variation of the positional relationships between the measurement point, the focal point F, and the receive aperture Rx. As a result, S/N ratio is effectively improved.

Moreover, two or more of one or more embodiments and the first and second setting examples in the present modification may be combined with each other. For example, the second target area Bx₂ may have a smaller inner angle at the focal point F than that of the first target area Bx₁, and have the maximum width in the transducer element array direction that is equal to or smaller than the width of the transmission aperture Tx. Alternatively, the second target area Bx₂ may have a smaller inner angle at the focal point F than that of the first target area Bx₁, and have the maximum width in the transducer element array direction that is equal to or smaller than that of the first target area Bx₁. Also, the second target area Bx₂ may for example be composed of a combination of an area that is close to the central axis Txo of the transmission aperture Tx and has a small inner angle at the focal point F and a linear area that is close to the outer boundary of the ultrasound main irradiation area Ax. As described above, the following methods are employed for reducing the number of measurement points in the second target area Bx₂: decrease in width of the second target area Bx₂ in the transducer element array direction; restriction of the maximum width of the second target area Bx₂ in the transducer element array direction; decrease in measurement point density in the transducer element array direction; and decrease in measurement point density in the transducer element array direction in areas distant from the central axis Txo of the transmission aperture Tx. These methods may be arbitrarily combined with each other.

In one or more embodiments described above, the second target area Bx₂ is set by the target area setter based on the transmission aperture Tx, the focal point F, and the ultrasound main irradiation area Ax. In one or more embodiments, the second target area Bx₂ is set based on ultrasound transmission and reception results.

An ultrasound diagnostic device pertaining to one or more embodiments described below differs from that pertaining to one or more embodiments described above only in terms of the method of setting the second target area Bx₂ by the target area setter and the configuration relating to the method. Thus, only the different points are described here, and description of other configurations and operations pertaining to one or more embodiments described below is not provided because of being the same as those in one or more embodiments described above.

### Configuration

The ultrasound diagnostic device pertaining to one or more embodiments includes a region setter in a control unit thereof.

The region setter generates an ultrasound image using an ultrasound probe and an ultrasound signal processing device, and notifies the target area setter of an area to be set as a target area based on the generated ultrasound image.

The region setter generates an ultrasound image using a transmission aperture Tx and a focal point F for a single transmission event and an ultrasound main irradiation area Ax relating to these. Specifically, the region setter performs a transmission event on the entirety of the ultrasound main irradiation area Ax as a temporary target area Bx₃ (test area) (Steps S101 and S102). The shape of the temporary target area Bx₃ may be arbitrary as long as the temporary target area Bx₃ includes the entirety of the ultrasound main irradiation area Ax. For example, the temporary target area Bx₃ may be a rectangle having the transmission aperture Tx as one of sides thereof. Next, the region setter performs beam forming on receive signal sequences for the transmission event. The details of beam forming are equivalent to combination of Steps S210 and 5220 or combination of Steps S210 and S420, and accordingly description thereof is omitted here. Then, the region setter sets a target area Bx based on sub-frame acoustic line signals acquired by beam forming.

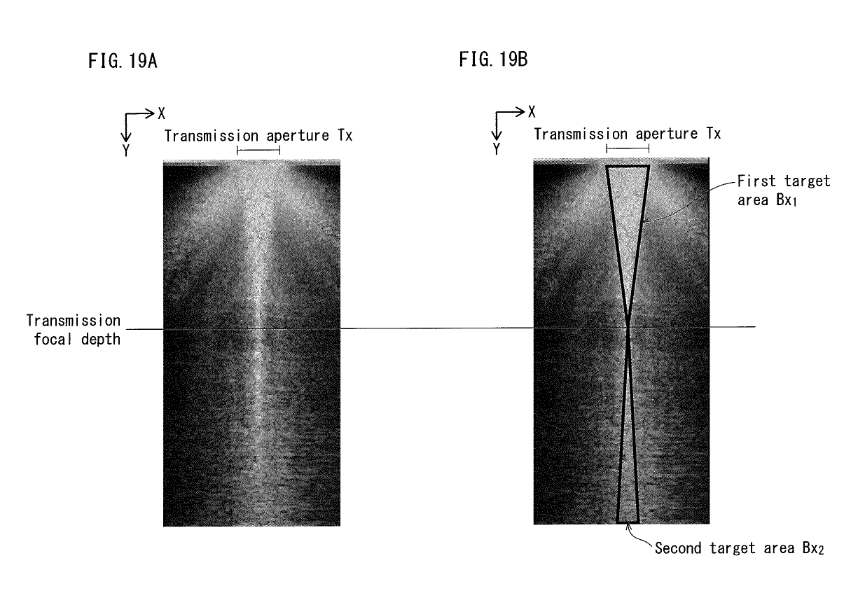

The following describes a method of setting the target area Bx based on sub-frame acoustic line signals. The region setter causes an ultrasound image generator to convert sub-frame acoustic line signals to an ultrasound image (B-mode image), and sets a target area Bx based on the ultrasound image thus generated (referred to in the following as an “evaluation image”). FIG. 19A illustrates an example of an evaluation image. Note that a target area Bx₃ is illustrated in FIG. 19A as being a rectangle having the entirety of the transducer element array of the ultrasound probe as one of sides thereof, in order to show ultrasound beam propagation. As illustrated in FIG. 19A, in areas at or shallower than the focal depth, a luminance value is high inside a triangle area defined by the focal point F and the transmission aperture Tx, and decreases outside the triangle area. Meanwhile, in areas deeper than the focal depth, the luminance value is high inside a triangle area that is narrower in the X direction than the triangle area, which is defined by the focal point F and the transmission aperture Tx, and decreases outside the narrower triangle area even inside the triangle area, which is defined by the focal point F and the transmission aperture Tx. This is due to directionality of transducer elements in ultrasound transmission and reception according to which amplitude of ultrasound beams and values of receive signals corresponding to ultrasound reflection both increase as approaching the central axis Txo of the transmission aperture Tx, and decrease as departing the central axis Txo. Accordingly, the region setter sets as the target area Bx, from among an evaluation image, an area having a luminance value equal to or greater a predetermined value. The predetermined value is for example a mean value of luminance values of an outer boundary of the triangle area, which is defined by the focal point F and the transmission aperture Tx, in areas shallower than the focal depth. The target area Bx set in this way is composed of a first target area Bx₁ and a second target area Bx₂ like in one or more embodiments, as illustrated in FIG. 19B. The first target area Bx₁ is a triangle area defined by the focal point F and the transmission aperture Tx. The second target area Bx₂ is a triangle area that is narrower in the X direction than the triangle area, which is defined by the focal point F and the transmission aperture Tx. Note that although sub-frame acoustic line signals are converted to an evaluation image here, the target area Bx may be set by comparing, with a predetermined value, an amplitude value of sub-frame acoustic line signals or an intensity value of reflected ultrasound extracted from the sub-frame acoustic line signals through envelope detection or the like.

The region setter sets the target area Bx prior to the start of the initial transmission event or prior to the start of the initial transmission event after change of the focal depth and/or the transmission aperture Tx. Then, the region setter causes the target region setter to use the set target area Bx.

With this configuration, the target area Bx includes only measurement points for which generated sub-frame acoustic line signals have a high S/N ratio, and excludes measurement points for generated sub-frame acoustic line signals have a low S/N ratio. Thus, it is possible to reduce computation amount to the maximum while maintaining S/N ratio of acoustic line signals to a standard or higher.

### Brief

According to the ultrasound diagnostic device pertaining to one or more embodiments, the second target area Bx₂, is set based on values of sub-frame acoustic line signals. This allows inclusion of, in the second target area, only measurement points for which S/N ratio of sub-frame acoustic line signals satisfies a certain standard in the ultrasound main irradiation area Ax. Therefore, it is possible to reduce computation amount to the maximum while maintaining S/N ratio of acoustic line signals to the certain standard or higher.

### Modification 3 of One or More Embodiments

In one or more embodiments, the description has been given on the case where the ultrasound diagnostic device actually performs ultrasound transmission and reception to and from the temporal target area Bx₃ (test area), and sets the target area Bx based on acoustic lines thus obtained.

However, the target area Bx is set based on characteristics of the ultrasound probe, characteristics of transmitted ultrasound beams, the transmission aperture Tx, and the focal depth. Therefore, the target area Bx can be set based on these parameters if they are known.

A region setter of an ultrasound diagnostic device pertaining to modification 3 stores therein a table showing correspondence among the characteristics of the ultrasound probe, the characteristics of transmitted ultrasound beams, the width of the transmission aperture Tx, the focal depth, and the target area Bx. The characteristics of the ultrasound probe indicate for example frequency characteristics of transducer elements, arrangement of transducer elements, and directionality of transducer elements in ultrasound transmission and reception. Note that the characteristics of the ultrasound probe may be, instead of a characteristic value itself, for example an ID identifying an ultrasound probe having predetermined characteristics, such as a model number of the ultrasound probe. The characteristics of transmitted ultrasound beams indicate for example frequency, amplitude, wave number, and transmission intervals of ultrasound. The region setter acquires the characteristics of the ultrasound probe from a control unit, the characteristics of the ultrasound probe and the width of the transmission aperture Tx from a transmission beam former, and causes a target region setter to set a target area Bx corresponding to these parameters.

Note that the region setter may store therein the above table in advance. With this configuration, it is possible to set an appropriate target area Bx without performing ultrasound transmission and reception to and from the test area. Also, in the case where the table includes no target area Bx that corresponds to the above parameters, the region setter may perform the operations described in the second one or more embodiments and add results of the operations to the table. With this configuration, in the case where no target area Bx exists, which corresponds to a combination of the characteristics of the ultrasound probe, the characteristics of transmitted ultrasound beams, the width of the transmission aperture Tx, and the focal depth, it is possible to set an appropriate target area Bx by performing ultrasound transmission and reception to and from the test area. Further, in the case where the table includes a target area Bx that corresponds to a combination of the characteristics of the ultrasound probe, the characteristics of transmitted ultrasound beams, the width of the transmission aperture Tx, and the focal depth, it is possible to use the corresponding target area Bx included in the table, thereby omitting ultrasound transmission and reception to and from the test area.

### Other Modifications

(1) In the one or more embodiments and the modifications, the number of measurement points included in the second target area Bx₂ is not especially defined. Alternatively, for example with respect to the number of measurement points included in the entirety of the target area Bx, the upper limit may be determined in accordance with computation capability of the delay-and-sum calculator and/or the synthesizer and/or the storage capacity of the data storage. Specifically, the size of the ultrasound main irradiation area Ax and the upper limit of a computation time period for one transmission event are determined in accordance with the frame rate of an ultrasound image, the width and depth of an ROI that is a target for which frame acoustic line signals are to be generated, the width of the transmission aperture Tx, and the shift pitch Mp. Compared with, the upper limit of the number of measurement points per time in the delay-and-sum calculator and the upper limit of the number of measurement points per time in the synthesizer are determined in accordance with hardware capability. Therefore, the target area may be set such that a time period necessary for computation does not exceed the upper limit of computation time period. Assume for example the case where the entirety of the ultrasound main irradiation area Ax is set as the target area Bx and the computation necessary time period is 1.25 times of the upper limit. In this case, the second target area Bx₂ is set such that the number of measurement points included in the target area Bx is equal to or less than 0.8 times of that in the case where the entirety of the ultrasound main irradiation area Ax is set as the target area Bx. Note that the second target area Bx₂ may be specifically set based on any one of one or more embodiments, and modifications 1 and 2. Also, in the case where the number of measurement points is still excessive after setting based on one or more embodiments or modification 3, the number of measurement points may be reduced based on any one of one or more embodiments, and modifications 1 and 2 of one or more embodiments. With this configuration of setting the second target area, it is possible to suppress frame dropping of ultrasound images due to an insufficient computation capability of the ultrasound signal processing device.

(2) In one or more embodiments and the modifications, the second target area Bx₂ is in a triangle shape having the focal point F as the vertex, a shape formed from a combination of a triangle and a rectangle, or a shape formed from straight lines. However, the shape of the second target area Bx₂ is not limited to the above ones. Alternatively, the second target area Bx₂ may be in a shape whose width in the transducer element array direction does not increase toward deeper areas. For example, the second target area Bx₂ may be in a shape formed from a combination of a triangle and a trapezoid. Further alternatively, the second target area Bx₂ may for example be a combination of a triangle area based on the luminance described in one or more embodiments or modification 3 and an area composed of straight lines described in the second setting example in modification 2.

(3) Up to this point, the present invention has been described based on the above one or more embodiments. However, the one or more embodiments described above are non-limiting examples of application of the present invention, and thus, the present invention shall be construed to encompass the following exemplar modifications.

For example, one or more embodiments of the present invention may be implemented by using a computer system including a memory storing a computer program and a microprocessor operating based on the computer program. For example, the computer system may store a computer program of the ultrasound signal processing method, and the computer system may operate in accordance with the computer program or may provide instructions in accordance with the computer program to various components connected thereto.

Further, one or more embodiments of the present invention may be implemented by implementing a part of or the entirety of the ultrasound signal processing device described above, or a part of or an entirety of a beam former described above by using a computer system including a microprocessor, a recording medium such as a ROM or a RAM, and a hard disk unit. In this implementation, a computer program achieving the same operations as a device described above is stored to the RAM or the hard disk unit. Further, in this implementation, various devices achieve their functions by the microprocessor operating in accordance with the computer program.

Further, one or more embodiments of the present invention may be implemented by implementing some or all components included in a device described above by using one system LSI (large scale integration). A system LSI is an ultra-multifunctional LSI manufactured by integrating multiple components onto one chip. Specifically, a system LSI is a computer system including a microprocessor, a ROM, a RAM, and the like. Further, each component may be separately implemented by using one chip, or some or all components may be implemented by using one chip. Note that LSIs are referred to by using different names, depending upon the level of integration achieved thereby. Such names include IC, system LSI, super LSI, and ultra LSI. In this implementation, a computer program achieving the same operations as any device described above is stored to the RAM. Further, in this implementation, the system LSI achieves its functions by the microprocessor operating in accordance with the computer program. For example, one or more embodiments of the present invention encompass a form of implementation where an LSI stores a beam forming method as a program, the LSI is inserted into a computer, and the computer executes the program (i.e., the beam forming method).

Note that integration of circuits may be achieved by a dedicated circuit or a general purpose processor, in addition to being achievable by using an LSI as discussed above. Further, a Field Programmable Gate Array (FPGA), which is programmable after manufacturing, or a reconfigurable processor, which allows reconfiguration of the connection and setting of circuit cells inside the LSI, may be used.

Furthermore, if technology for circuit integration that replaces LSIs emerges, owing to advances in semiconductor technology or to another derivative technology, the integration of functional blocks may naturally be accomplished using such technology.

Further, some or all functions of an ultrasound diagnostic device discussed in the above one or more embodiments may be implemented by a processor such as a CPU executing a program. Further, one or more embodiments of the present invention may be implemented by using a non-transitory computer-readable recording medium having recorded thereon a program causing execution of a diagnostic method and a beam forming method of an ultrasound diagnostic device. Further, execution of the program by another independent computer system may be achieved by transferring the program by recording the program or a signal onto a recording medium. Naturally, the program may be distributed via means of transmission media such as the internet.

The ultrasound diagnostic device pertaining to the one or more embodiments includes the data storage, which is a recording device. However, the recording device need not be included in the ultrasound diagnostic device, and may be implemented by using a semiconductor memory, a hard disk drive, an optical disk drive, a magnetic storage device, or the like connected to the ultrasound diagnostic device from the outside.

Further, the functional blocks illustrated in the block diagrams are mere examples of possible functional blocks. That is, a plurality of functional blocks illustrated in the block diagrams may be combined to form one functional block, a given functional block illustrated in the block diagrams may be divided into a plurality of functional blocks, and a function of a given functional block illustrated in the block diagrams may be transferred to another functional block. Further, with regards to multiple functional blocks having similar functions, such functional blocks may be implemented by one piece of hardware or software executing such functions in parallel or by applying time division.

Further, the above-described order in which steps of processing are executed is a non-limiting example among multiple possible orders that is used for the sole sake of providing specific description of one or more embodiments of the present invention. Further, some of the steps of processing described above may be executed simultaneously (in parallel).

Further, in one or more embodiments, description is provided that the ultrasound diagnostic device may have a probe and a display attached thereto. However, the ultrasound diagnostic device may include a probe and a display therein.

Further, in one or more embodiments, the probe includes a plurality of piezoelectric transducer elements forming a line in one direction. However, the probe may have a different structure. For example, the probe may include a plurality of piezoelectric transducer elements disposed two-dimensionally. Alternatively, the probe may be a swingable probe including a plurality of swingable transducer elements (i.e., transducer elements that can be caused to swing by mechanical means) forming a line in one direction, which enables acquisition of three-dimensional tomographic images. Further, probes of different types may be selected and used depending upon the examination to be performed. For example, when using a probe including piezoelectric transducer elements disposed two-dimensionally, supplying different piezoelectric transducer elements with voltages at different timings or with voltages with different values achieves controlling the position, the direction, etc., of the ultrasound beam to be transmitted.

Further, the probe may be provided with some of the functions of the transmission beam former/receive beam former. For example, the probe may be capable of generating a transmission electric signal based on a control signal that the transmission beam former/receive beam former outputs to cause generation of a transmission electric signal, and of converting the transmission electronic signal into ultrasound. In addition, the probe may be capable of converting reflected ultrasound into a receive electric signal, and of generating a receive signal based on the receive electric signal.

Further, at least some of the functions of the ultrasound diagnostic devices pertaining to one or more embodiments and the modification may be combined with functions of other ones of the ultrasound diagnostic devices pertaining to the one or more embodiments and the modifications. Further, the values used above are non-limiting examples used for the sole sake of providing specific description of one or more embodiments of the present invention, and may be replaced with other values.

Further, the present invention should be construed as encompassing various modifications that a skilled artisan would arrive at based on one or more embodiments described above.

### Brief

(1) One or more embodiments of the present invention are ultrasound signal processing devices that performs multiple transmission events of transmitting converging ultrasound beams to a subject by using an ultrasound probe having multiple transducer elements, that performs, for each of the transmission events, reception of ultrasound reflection from the subject and generation of a sub-frame acoustic line signal based on the ultrasound reflection, and that synthesizes sub-frame acoustic line signals for the respective transmission events to generate a frame acoustic line signal, the ultrasound signal processing device comprising ultrasound signal processing circuitry configured to operate as: a transmitter that, for each of the transmission events, while shifting a transmission transducer element array of the ultrasound probe in a transducer element array direction in which the transducer elements are arrayed, causes the ultrasound probe to transmit ultrasound beams to an ultrasound main irradiation area by using the transmission transducer element array, the ultrasound beams converging at a focal point defined by a position of the transmission transducer element array, the ultrasound main irradiation area being defined as an area positioned between two straight lines each connecting the focal point and a different end of the transmission transducer element array; a receiver that, for each of the transmission events, generates sequences of receive signals for receive transducer elements of the ultrasound probe based on ultrasound reflection that the ultrasound probe receives from the subject; a delay-and-sum calculator that, for each of the transmission events, sets a first target area and a second target area included in the ultrasound main irradiation area, and performs delay-and-summing of sequences of receive signals based on ultrasound reflection from measurement points located in the first target area and the second target area thereby to generate a sub-frame acoustic line signal for each of the measurement points, the first target area being an entirety of an area located at or shallower than a focal depth where the focal point is located, the second target area being part of an area located deeper than the focal depth; and a synthesizer that synthesizes sub-frame acoustic line signals for the transmission events to generate a frame acoustic line signal.

Also, one or more embodiments of the present invention are ultrasound signal processing methods of performing multiple transmission events of transmitting converging ultrasound beams to a subject by using an ultrasound probe having multiple transducer elements, performing, for each of the transmission events, reception of ultrasound reflection from the subject and generation of a sub-frame acoustic line signal based on the ultrasound reflection, and synthesizing sub-frame acoustic line signals for the respective transmission events to generate a frame acoustic line signal, the ultrasound signal processing method comprising: for each of the transmission events, while shifting a transmission transducer element array of the ultrasound probe in a transducer element array direction in which the transducer elements are arrayed, causing the ultrasound probe to transmit ultrasound beams to an ultrasound main irradiation area by using the transmission transducer element array, the ultrasound beams converging at a focal point defined by a position of the transmission transducer element array, the ultrasound main irradiation area being defined as an area positioned between two straight lines each connecting the focal point and a different end of the transmission transducer element array; for each of the transmission events, generating sequences of receive signals for receive transducer elements of the ultrasound probe based on ultrasound reflection that the ultrasound probe receives from the subject; for each of the transmission events, setting a first target area and a second target area included in the ultrasound main irradiation area, and performing delay-and-summing of sequences of receive signals based on ultrasound reflection from measurement points located in the first target area and the second target area thereby to generate a sub-frame acoustic line signal for each of the measurement points, the first target area being an entirety of an area located at or shallower than a focal depth where the focal point is located, the second target area being part of an area located deeper than the focal depth; and synthesizing sub-frame acoustic line signals for the transmission events to generate a frame acoustic line signal.

With the above configuration or method, it is possible to reduce the number of measurement points while suppressing decrease in spatial resolution and S/N ratio of frame acoustic line signals, thereby reducing computation amount for delay-and-summing taking into consideration transmission and reception delays and for synthesizing.

(2) Also, in the ultrasound signal processing device in the above section (1), the second target area, at a range from the focal depth to a depth twice the focal depth, may include a smaller number of measurement points than the first target area.

With the above configuration, it is possible to set a higher average measurement point density in the first target area than in the second target area, thereby suppressing degradation of acoustic line signal quality in areas shallower than the focal depth.

(3) Also, in the ultrasound signal processing device in the above section (1) or (2), the second target area may include a smaller number of measurement points per unit of area than the first target area.

With the above configuration, it is possible to set a lower measurement point density in the entirety of the second target area than in the first target area, thereby ensuring reduction in computation amount.

(4) Also, in the ultrasound signal processing device in the above sections (1)-(3), the first target area and the second target area may each have a shape whose vertex coincides with the focal point, and may be each symmetric with respect to a straight line that is perpendicular to the transducer element array direction and passes through the focal point, and the second target area may have a smaller inner angle at the focal point as the vertex than the first target area.

With the above configuration, it is possible to set, as the second target area, an area that is close to the central axis of the transmission aperture, thereby suppressing decrease in S/N ratio of acoustic line signals.

(5) Also, in the ultrasound signal processing device in the above sections (1)-(4), the second target area may have a maximum width in the transducer element array direction that is equal to or smaller than a width of the transmission transducer element array.

With the above configuration, even in the case where the maximum depth of the second target area is large relative to the focal depth, it is possible to suppress increase in the number of measurement points located in the second target area due to increase in size of the second target area.

(6) Also, in the ultrasound signal processing device in the above section (5), the second target area may have the smaller maximum width in the transducer element array direction than the first target area.

With the above configuration, it is possible to further restrict the size of the second target area, thereby reducing computation amount.

(7) Also, in the ultrasound signal processing device in the above sections (1)-(6), the second target area may be composed of a plurality of linear areas each passing through the focal point, and any measurement point, on any of the linear areas, that is spaced away from the focal point by a predetermined distance or more may satisfy a condition that a distance between the measurement point and a most nearby measurement point on the same linear area is smaller than a distance between the measurement point and a most nearby one among measurement points on an adjacent linear area.

With the above configuration, it is possible to reduce the number of measurement points by decreasing the measurement point density in the transducer element array direction while maintaining a wide variation range of ultrasound beam travel directions and a large variation among transmission events of the positional relationships between the measurement point, the focal point F, and the receive aperture. Therefore, it is possible to further suppress the degree of decrease in spatial resolution and S/N ratio of frame acoustic line signals relative to a reduction amount of computation amount.

(8) Also, in the ultrasound signal processing device in the above sections (1)-(7), part of the second target area may include measurement points whose density in the transducer element array direction increases as approaching a straight line that is perpendicular to the transducer element array direction and passes through the focal point.

With the above configuration, it is possible to suppress decrease in S/N ratio of acoustic line signals as the measurement point density increases with respect to areas for which a higher S/N ratio of acoustic line signals is acquired.

(9) Also, the ultrasound signal processing device in the above sections (1)-(8) may further comprise an area setter that sets an ultrasound main irradiation area inside the subject, sets a focal point based on the ultrasound main irradiation area, controls the transmitter to cause the ultrasound probe to transmit ultrasound beams for convergence at the focal point, controls the receiver to generate sequences of receive signals based on ultrasound reflection corresponding to the ultrasound beams, sets a plurality of measurement points in a test area including the ultrasound main irradiation area, controls the delay-and-sum calculator to generate a sub-frame acoustic line signal for each of the measurement points, and sets a first target area and a second target area based on sub-frame acoustic line signals for the measurement points.

With the above configuration, it is possible to set the second target area based on actual measured values of S/N ratio of acoustic line signals. Thus, it is possible to appropriately set measurement points necessary for the S/N ratio of acoustic line signals to satisfy a user's standard, thereby minimizing computation amount within a range that acoustic line signal quality satisfies the user's standard.

(10) Also, in the ultrasound signal processing device in the above section (9), the area setter may set as the first target area and the second target area, from among the test area, an area including measurement points corresponding to sub-frame acoustic line signals having an amplitude equal to or greater than a predetermined threshold value.

With the above configuration, it is possible to set the second target area by performing simple processing based on acoustic line signals.

(11) Also, the ultrasound signal processing device in the above sections (1)-(8) may further comprise an area setter that sets the first target area and the second target area using characteristics of the ultrasound probe.

With the above configuration, it is possible to estimate positional dependence of S/N ratio of acoustic line signals based on the characteristics of the ultrasound probe, thereby setting an appropriate second target area.

(12) Also, the ultrasound signal processing device in the above section (11) may further comprise an ultrasound probe characteristics storage that stores therein characteristics for each ultrasound probe, wherein the area setter may acquire the characteristics of the ultrasound probe used in the ultrasound signal processing device from the ultrasound probe characteristics storage.

With the above configuration, it is possible to set an appropriate second target area for each ultrasound probe to be used.

(13) Also, in the ultrasound signal processing device in the above sections (1)-(12), the second target area may be set such that the total number of measurement points located in the first target area and the second target area does not exceed a predetermined upper limit that is determined by the delay-and-sum calculator and the synthesizer.

With the above configuration, it is possible to suppress the number of measurement points within a range of processing capability of the ultrasound signal processing device, thereby suppressing failures such as a so-called frame dropping due to an insufficient processing capability.

An ultrasound signal processing device, an ultrasound diagnostic device, and an ultrasound signal processing method pertaining to one or more embodiments of the present invention are useful in improving the performance of conventional ultrasound diagnostic devices, and in particular, are useful in reducing computation device cost and in improving frame rate through reduction in computation load. In addition, the present invention, as well as being applicable to ultrasound, is also applicable for example to sensors having array elements.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. An ultrasound signal processing device that performs multiple transmission events of transmitting converging ultrasound beams to a subject by using an ultrasound probe having multiple transducer elements, that performs, for each of the transmission events, reception of ultrasound reflection from the subject and generation of a sub-frame acoustic line signal based on the ultrasound reflection, and that synthesizes sub-frame acoustic line signals for the respective transmission events to generate a frame acoustic line signal, the ultrasound signal processing device comprising:
an ultrasound signal processing circuitry that operates as:
a transmitter that, for each of the transmission events:
while shifting a transmission transducer element array of the ultrasound probe in a transducer element array direction in which the transducer elements are arrayed, causes the ultrasound probe to transmit ultrasound beams to an ultrasound main irradiation area by using the transmission transducer element array,
wherein the ultrasound beams converging at a focal point defined by a position of the transmission transducer element array, and
wherein the ultrasound main irradiation area is defined as an area positioned between two straight lines each connecting the focal point and a different end of the transmission transducer element array;
a receiver that, for each of the transmission events, generates sequences of receive signals for receive transducer elements of the ultrasound probe based on ultrasound reflection that the ultrasound probe receives from the subject;
a delay-and-sum calculator that, for each of the transmission events:
sets a first target area and a second target area included in the ultrasound main irradiation area, and
performs delay-and-summing of sequences of receive signals based on ultrasound reflection from measurement points located in the first target area and the second target area thereby to generate a sub-frame acoustic line signal for each of the measurement points,
wherein the first target area is an entirety of an area located at or shallower than a focal depth where the focal point is located, and
wherein the second target area is part of an area located deeper than the focal depth; and
a synthesizer that synthesizes sub-frame acoustic line signals for the transmission events to generate a frame acoustic line signal.

2. The ultrasound signal processing device of claim 1, wherein
the second target area, at a range from the focal depth to a depth twice the focal depth, includes a smaller number of measurement points than the first target area.

3. The ultrasound signal processing device of claim 1, wherein
the second target area includes a smaller number of measurement points per unit of area than the first target area.

4. The ultrasound signal processing device of claim 1, wherein
the first target area and the second target area each have a shape whose vertex coincides with the focal point, and are each symmetric with respect to a straight line that is perpendicular to the transducer element array direction and passes through the focal point, and
the second target area has a smaller inner angle at the focal point as the vertex than the first target area.

5. The ultrasound signal processing device of claim 1, wherein
the second target area has a maximum width in the transducer element array direction that is equal to or smaller than a width of the transmission transducer element array.

6. The ultrasound signal processing device of claim 5, wherein
the second target area has the smaller maximum width in the transducer element array direction than the first target area.

7. The ultrasound signal processing device of claim 1, wherein
the second target area is composed of a plurality of linear areas that each passes through the focal point, and any measurement point, on any of the linear areas, that is spaced away from the focal point by a predetermined distance or more satisfies a condition that a distance between the measurement point and a most nearby measurement point on the same linear area is smaller than a distance between the measurement point and a most nearby one among measurement points on an adjacent linear area.

8. The ultrasound signal processing device of claim 1, wherein
part of the second target area includes measurement points whose density in the transducer element array direction increases as approaching a straight line that is perpendicular to the transducer element array direction and passes through the focal point.

9. The ultrasound signal processing device of claim 1, further comprising an area setter that:
sets an ultrasound main irradiation area inside the subject,
sets a focal point based on the ultrasound main irradiation area,
controls the transmitter to cause the ultrasound probe to transmit ultrasound beams for convergence at the focal point,
controls the receiver to generate sequences of receive signals based on ultrasound reflection corresponding to the ultrasound beams,
sets a plurality of measurement points in a test area including the ultrasound main irradiation area,
controls the delay-and-sum calculator to generate a sub-frame acoustic line signal for each of the measurement points, and
sets a first target area and a second target area based on sub-frame acoustic line signals for the measurement points.

10. The ultrasound signal processing device of claim 9, wherein
the area setter sets as the first target area and the second target area, from among the test area, an area including measurement points corresponding to sub-frame acoustic line signals having an amplitude equal to or greater than a predetermined threshold value.

11. The ultrasound signal processing device of claim 1, further comprising:
an area setter that sets the first target area and the second target area using characteristics of the ultrasound probe.

12. The ultrasound signal processing device of claim 11, further comprising:
an ultrasound probe characteristics storage that stores therein characteristics for each ultrasound probe, wherein
the area setter acquires the characteristics of the ultrasound probe used in the ultrasound signal processing device from the ultrasound probe characteristics storage.

13. The ultrasound signal processing device of claim 1, wherein
the second target area is set such that the total number of measurement points located in the first target area and the second target area does not exceed a predetermined upper limit that is determined by the delay-and-sum calculator and the synthesizer.

14. An ultrasound diagnostic device that performs multiple transmission events of transmitting converging ultrasound beams to a subject by using an ultrasound probe having multiple transducer elements, that performs, for each of the transmission events, reception of ultrasound reflection from the subject and generation of a sub-frame acoustic line signal based on the ultrasound reflection, and that synthesizes sub-frame acoustic line signals for the respective transmission events to generate a frame acoustic line signal, the ultrasound diagnostic device comprising:
the ultrasound probe; and
ultrasound signal processing circuitry, wherein
the ultrasound signal processing circuitry operates as:
a transmitter that, for each of the transmission events:
while shifting a transmission transducer element array of the ultrasound probe in a transducer element array direction in which the transducer elements are arrayed, causes the ultrasound probe to transmit ultrasound beams to an ultrasound main irradiation area by using the transmission transducer element array,
wherein the ultrasound beams converging at a focal point defined by a position of the transmission transducer element array, and
wherein the ultrasound main irradiation area is defined as an area positioned between two straight lines each connecting the focal point and a different end of the transmission transducer element array;
a receiver that, for each of the transmission events, generates sequences of receive signals for receive transducer elements of the ultrasound probe based on ultrasound reflection that the ultrasound probe receives from the subject;
a delay-and-sum calculator that, for each of the transmission events:
sets a first target area and a second target area included in the ultrasound main irradiation area, and
performs delay-and-summing of sequences of receive signals based on ultrasound reflection from measurement points located in the first target area and the second target area thereby to generate a sub-frame acoustic line signal for each of the measurement points,
wherein the first target area is an entirety of an area located at or shallower than a focal depth where the focal point is located, and
wherein the second target area is part of an area located deeper than the focal depth; and
a synthesizer that synthesizes sub-frame acoustic line signals for the transmission events to generate a frame acoustic line signal.

15. An ultrasound signal processing method of performing multiple transmission events of transmitting converging ultrasound beams to a subject by using an ultrasound probe having multiple transducer elements, performing, for each of the transmission events, reception of ultrasound reflection from the subject and generation of a sub-frame acoustic line signal based on the ultrasound reflection, and synthesizing sub-frame acoustic line signals for the respective transmission events to generate a frame acoustic line signal, the ultrasound signal processing method comprising:
for each of the transmission events, while shifting a transmission transducer element array of the ultrasound probe in a transducer element array direction in which the transducer elements are arrayed:
causing the ultrasound probe to transmit ultrasound beams to an ultrasound main irradiation area by using the transmission transducer element array,
wherein the ultrasound beams converging at a focal point defined by a position of the transmission transducer element array, and
wherein the ultrasound main irradiation area is defined as an area positioned between two straight lines each connecting the focal point and a different end of the transmission transducer element array;
for each of the transmission events:
generating sequences of receive signals for receive transducer elements of the ultrasound probe based on ultrasound reflection that the ultrasound probe receives from the subject;
setting a first target area and a second target area included in the ultrasound main irradiation area, and
performing delay-and-summing of sequences of receive signals based on ultrasound reflection from measurement points located in the first target area and the second target area thereby to generate a sub-frame acoustic line signal for each of the measurement points,
wherein the first target area is an entirety of an area located at or shallower than a focal depth where the focal point is located, and
wherein the second target area is part of an area located deeper than the focal depth; and
synthesizing sub-frame acoustic line signals for the transmission events to generate a frame acoustic line signal.
